(19) United States
(12) Patent Application Publication
(10) Pub. No.: US 2017/0281946 A1
(43) Pub. Date: Oct. 5, 2017

(54) SYSTEMS AND METHODS FOR PATIENT REHABILITATION USING BRAIN STIMULATION

(71) Applicant: The General Hospital Corporation, Boston, MA (US)

(72) Inventors: Husam Katnani, Nahant, MA (US); Emad N. Eskandar, Boston, MA (US)

(21) Appl. No.: 15/509,437

(22) PCT Filed: Sep. 28, 2015
(86) PCT No.: PCT/US15/52570

§ 371 Date: Mar. 7, 2017

Related U.S. Application Data
(60) Provisional application No. 62/056,096, filed on Sep. 26, 2014.
(60) Provisional application No. 62/157,704, filed on May 6, 2015.

(51) Int. Cl. A61N 1/36; A61N 1/02; A61N 1/05

(52) U.S. Cl. CPC A61N 1/36103; A61N 1/0531; A61N 1/0534; A61N 1/36192; A61N 1/36196; A61N 1/36132; A61N 1/025; A61N 1/36082; A61N 1/36067

(57) ABSTRACT

Systems and methods are provided for stimulating the brain of a patient to treat a medical condition. In some aspects, a method includes positioning a stimulating device comprising electrical contacts configured to electrically stimulate locations associated with a patient's brain, and initiating a rehabilitation process to include the patient performing a task. The method also includes acquiring feedback from the patient at least while the patient is performing the task, generating, based on the acquired feedback, electrical stimulations to treat the medical condition of the patient. In some aspects, the method further includes generating a report indicative of a patient performance.

### CROSS-REFERENCE TO RELATED APPLICATIONS

This application is based on, and incorporates herein in their entirety, U.S. Provisional Patent Application Ser. No. 62/056,096 filed on Sep. 26, 2014 and entitled “SYSTEMS AND METHODS FOR PATIENT REHABILITATION USING BRAIN STIMULATION,” and U.S. Provisional Patent Application Ser. No. 62/157,704 filed on May 6, 2015 and entitled “WIDESPREAD STIMULATION-INDUCED CELLULAR ADAPTATION FOR NEURAL RECOVERY FOLLOWING BRAIN INJURY.”

### FIELD OF THE INVENTION

The present invention relates to systems and methods for patient rehabilitation and, more particularly, to a system and method for using electrical stimulation of brain structures to rehabilitate a patient suffering from neurological disorders caused by an injury or medical condition.

### BACKGROUND OF THE INVENTION

Brain injuries such as stroke and traumatic injury, as well as disorders including Alzheimer's disease (“AD”), dementia and autism represent major public health concerns. For instance, the CDC estimates a nationwide prevalence of stroke to be about 2.6% or roughly 5,839,000 patients, with a cost of treatment estimated at $62.7 billion in 2007. In addition, traumatic brain injury also affects a large number of patients in the United States, estimated by the CDC to be about 1,400,000 patients per year.

Presently, treatments for brain conditions or disorders are largely supportive in nature. In the case of stroke or temporary brain injury (“TBI”), treatment is commonly consists of two phases, namely an acute treatment phase followed by a period of rehabilitation. The acute treatment is generally concerned with weathering the immediate conditions, and preventing secondary injury due to brain edema, hemorrhage, seizure, and/or other complications. The rehabilitation phase includes a regimen of behavioral and/or physical therapy in order to recuperate cognitive and motor deficits caused by injury.

Despite advances in the understanding of the pathophysiological damage that occurs following TBI and stroke, current post-injury rehabilitation approaches are limited. Secondary injuries cause significant harm by spreading cellular damage that can grow to encompass a much greater area of the brain than was originally impacted. Following injury, widespread loss of cerebral connectivity at the cellular level is assumed to underlie the failure of neural processing at the systems level that supports communication and goal-directed behavior, thus causing cognitive and motor deficits. Specifically, different regions of the cortico-striato-thalamo-cortical (“CSTC”) circuits associated with critical motor and cognitive function exhibit attenuated neural signals and abnormal oscillatory firing patterns. Although converging evidence suggests that there may be some cortical plasticity following brain injury, for instance mediated through striatal connections, rehabilitation via behavioral and/or physical therapy is slow, imperfect, and may not be easily accessible. In addition, it is not clear that patients reach their maximum potential recovery.

In some attempts, deep brain stimulation (“DBS”) systems have been used to treat various neurological disorders, including movement disorders such as Parkinson Disease and Essential Tremor. However, in spite of the enormous strides in electrical engineering technology, commercial DBS systems, akin to cardiac pacemakers, have not fundamentally changed for over two decades and are limited in flexibility. For example, conventional DBS systems generally include a small number of channels and operate in an “open-loop” fashion, where stimulation is delivered to the patient's brain continuously or according to a pre-determined algorithm regardless of the patient's current status or progress. Thus, available DBS systems are unable to monitor the status or progress of a patient, and have no extrinsic or intrinsic feedback control to provide optimum care for a patient. Furthermore, such systems cannot be used to treat traumatic brain injury, stroke, AD, autism, and many other disorders.

Hence, there is a need for systems and methods directed to patient rehabilitation or therapeutic treatment via brain stimulation tailored to the particular medical needs and progress of each patient.

### SUMMARY OF THE INVENTION

The present disclosure overcomes drawbacks of previous technologies by providing systems and methods for treating or rehabilitating cognitive and/or motor deficits due to neurological disorders. More specifically, the present disclosure describes systems and methods that implement a novel closed-loop approach utilizing brain stimulation in conjunction with behavioral tasks while taking into account patient feedback, for purposes including enhanced learning, motivation and/or memory formation. In some applications, selective electrical stimulations triggered at specific time points during task performance may be utilized to treat patients with specific medical conditions, such as patients in recovery from traumatic brain injury (“TBI”) or stroke.

In one aspect of the present disclosure, a method for stimulating the brain of a patient to treat a medical condition is provided. The method positioning a stimulating device comprising electrical contacts configured to electrically stimulate a plurality of locations in a patient's brain, and initiating a rehabilitation process to include the patient performing a task. The method also includes providing, using the stimulating device, a first electrical stimulation to a first location in the patient's brain, the first electrical stimulation occurring at a first time point during the task, and acquiring, using a capture system, feedback from the patient while the patient is performing the task. The method further includes providing, using the acquired feedback, a second electrical stimulation to a second location in the patient's brain, the second electrical stimulation occurring at a second time point relative to the first time point.

In another aspect of the present disclosure, a method for stimulating the brain of a patient for treating a condition is provided. The method includes positioning a stimulating device comprising electrical contacts configured to electrically stimulate locations associated with a patient's brain, and initiating a rehabilitation process to include the patient performing a task. The method also includes acquiring feedback from the patient at least while the patient is performing the task, generating, based on the acquired feedback, electrical stimulations to treat the medical condition of the patient. In some aspects, the method further includes generating a report indicative of a patient performance.

In yet another aspect of the present disclosure, a system for stimulating the brain of a patient to treat a medical condition is provided. The system includes a stimulation system comprising electrical contacts configured to electrically stimulate locations associated with a patient's brain, and a capture system, in communication with the stimulation system, comprising an input configured to receive feedback from the patient, and a processor. The processor is at least configured to initiate a rehabilitation process to include the patient performing a task, and acquire, using the input, feedback from the patient. The processor is also configured to generate an electrical stimulation based on the acquired feedback, and trigger the stimulation system to deliver the electrical stimulation to treat the medical condition of the patient.

The foregoing and other aspects and advantages of the invention will appear from the following description. In the description, reference is made to the accompanying drawings which form a part hereof, and in which there is shown by way of illustration at least one embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is an image of a coronal section of a brain showing the caudate nucleus and nucleus accumbens.

FIG. 2A is a schematic diagram for a closed-loop brain stimulation system, in accordance with aspects of the present disclosure.

FIG. 2B is an illustration showing one embodiment of the closed-loop brain stimulation system of FIG. 2A.

FIG. 3A is an illustration showing one embodiment of the implantable device shown in FIG. 2B.

FIG. 3B are schematic views illustrating one embodiment of the control module shown in FIG. 3A.

FIG. 3C is a block diagram illustrating one embodiment of the implantable device shown in FIG. 2B.

FIG. 3D is a block diagram illustrating one embodiment of the signal generation module shown in FIG. 3C.

FIG. 3E is a block diagram illustrating one embodiment of the communication module shown in FIG. 3C.

FIG. 4 is a block diagram illustrating one embodiment of the wearable device shown in FIG. 2B.

FIG. 5 is a flowchart setting forth steps of a process for closed-loop electrical stimulation, in accordance with aspects of the present disclosure.

FIG. 6 is flowchart setting forth steps of another process for closed-loop electrical stimulation, in accordance with aspects of the present disclosure.

FIG. 7A is a schematic diagram illustrating an example treatment process based on electrical stimulations to multiple brain tissues, in accordance with aspects of the present disclosure.

FIG. 7B is a schematic diagram showing an example task-based rehabilitation process, in accordance with aspects of the present disclosure.

FIG. 8A is a graph comparing learning performance for animal subjects treated using electrical stimulations in accordance with aspects of the present disclosure.

FIG. 8B is a graph showing state-space approach learning curves for animal subjects treated using electrical stimulations in accordance with aspects of the present disclosure.

FIG. 8C is a graph showing the distribution of learning for animal subjects treated using electrical stimulations in accordance with aspects of the present disclosure.

FIG. 8D shows a scatter plot of reaction time for animal subjects treated using electrical stimulations in accordance with aspects of the present disclosure.

FIG. 9 is an illustration comparing tracking patterns during a learning process for un-stimulated animal subjects, and animal subjects stimulated in accordance with aspects of the present disclosure.

FIG. 10A is an image of a hippocampus coronal section showing cerebral damage due to impact.

FIG. 10B is a graph showing wire grip testing scores comparing control subjects and subjects post-injury.

FIG. 10C is an illustration showing the placement of lead contacts and brain sections removed for transcriptome tissue preparation.

FIG. HA is a graph comparing escape latencies between untreated subjects and subjects treated in accordance with aspects of the present disclosure after 5 days of testing.

FIG. 11B is another graph comparing escape latencies between untreated subjects and subjects treated in accordance with aspects of the present disclosure after 12 days of testing.

FIG. 11C is yet another graph comparing escape latencies between untreated subjects and subjects treated in accordance with aspects of the present disclosure after 19 days of testing.

FIG. 12A is a graphical illustration showing a top down view of spatial exploration taken across 12 days of testing for a control group.

FIG. 12B is a graphical illustration showing a top down view of spatial exploration taken across 12 days of testing for an untreated animal group.

FIG. 12C is a graphical illustration showing a top down view of spatial exploration taken across 12 days of testing for an animal group treated in accordance with aspects of the present disclosure.

FIG. 12D is a graph showing path efficiency in animal groups measured in FIGS. 12A, 12B and 12C.

FIG. 13A is a location plot showing real-time place preference for a representative animal subject.

FIG. 13B is a graph showing the effect of task stimulation parameters on hedonic response for different groups of animal subjects.

FIG. 13C is a graph showing distance traveled for different groups of animal subjects.

FIG. 14 are graphs comparing escape latency and path efficiency across different groups of animal subjects.

FIG. 15A are images showing example gene expression across different groups of animal subjects.

FIG. 15B is a graph showing results from a quantitative analysis of gene expression in the ipsilateral subventricular zone and rostral migratory stream of different groups of animal subjects.

FIG. 15C is another graph showing a comparison of labeled cells found in ipsilesional and contralesional nucleus accumbens and hippocampus.

FIG. 16 is a yet another graph showing a comparison of fragment per kilobase of transcript per million mapped for different groups of animal subjects.

FIG. 17 is a graphical illustration demonstrating that injury and stimulation, in accordance with aspects of the present disclosure, can cause broad changes in gene expression.

FIG. 18 are graphs demonstrating that stimulation, in accordance with aspects of the present disclosure, can alter differential expression of marker genes.

### DETAILED DESCRIPTION

The present disclosure is directed to brain stimulation, and in particular to brain stimulation that takes into account patient feedback and performance. As will be appreciated from descriptions below, the present approach may find use in a wide range of applications, including treatment of medical conditions, enhanced learning, motivation, memory formation, and so forth. In some aspects, systems and methods described may be applied to patient rehabilitation, maximizing neural and cognitive recovery and improving functional outcomes. For instance, important applications include the treatment of traumatic brain injury (“TBI”), stroke, as well as other conditions.

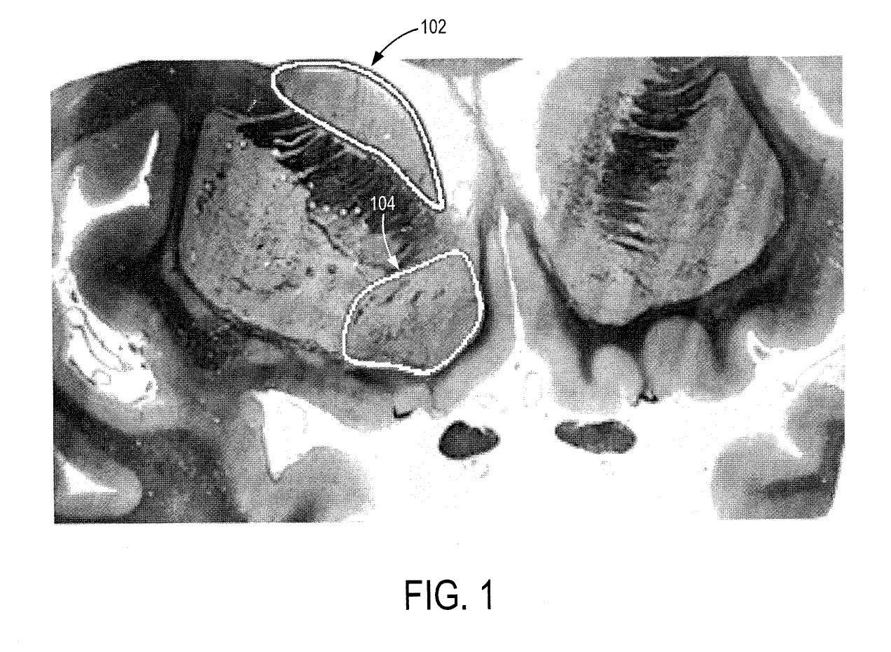

The present disclosure relates in part to a discovery by the inventors that administration of electrical stimulation to certain brain tissues during a learning interval of a behavioral learning task, specifically the reinforcement interval, can increase the rate of learning by a patient performing the task. In particular, results established that providing electrical stimulation to specific portions of the brain, namely the caudate (“Cd”) nucleus, during a controlled window of time can enhance memory formation and retention. By way of example, FIG. 1 depicts a coronal section of the brain showing the region containing the hippocampus, nucleus basalis, and mammillary bodies, and more specifically the Cd nucleus 102 and nucleus accumbens 104 are indicated.

As will be described, this approach may be extended to include electrical stimulation to other brain tissues for enhancement neural and cognitive function or recovery. For instance, the nucleus accumbens (“NAcc”) of the brain, which is implicated in motivation, memory and reward-based learning, may be also subjected to electrical stimulation. As will be shown, stimulation of the NAcc can induce widespread cellular adaptation to increase neuronal precursor cells and synaptic density as well as regulate marker genes associated with neural differentiation and migration, cell signaling and neuroprotection, providing a major advance in demonstrating a viable therapy for functional recovery of deficits, for instance, following brain injury.

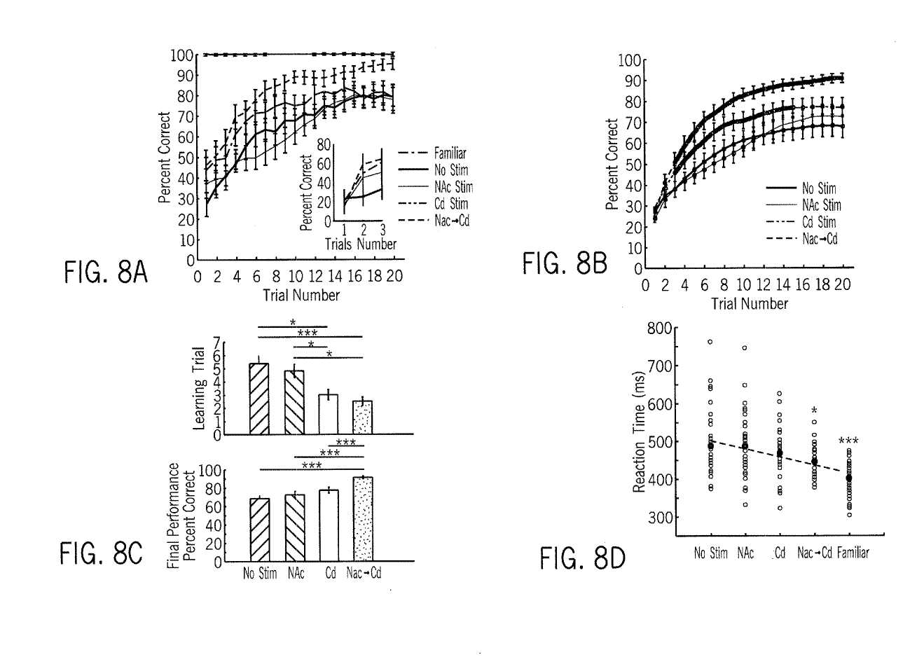

In fact, it is a discovery of the present disclosure that electrical stimulation applied to multiple brain tissues, in appropriate relative timing, can provide results not expected from separate stimuli. Specifically, appropriately timed NAcc stimulation in relation to stimulation of the Cd, may be performed in order to further enhance learning, motivation and association formations. As detailed below, it is a surprising finding of the present disclosure that electrical stimulation of the NAcc applied at the start interval of a task, combined with electrical stimulation of the Cd applied at the reinforcement interval of a task, enhances the performance of a patient well beyond stimulation of the Cd alone. By comparison, electrical stimulation of the NAcc alone does not generate a similar result, nor does combining electrical stimulation of the NAcc and the Cd at different intervals, as may be appreciated from FIGS. 8A-8D which show learning performance for the above-described electrical stimulations.

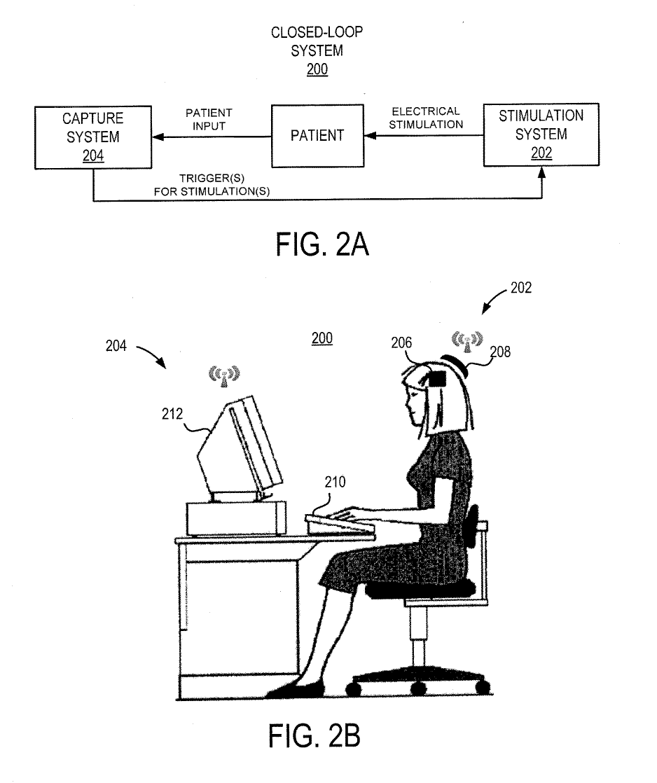

Turning now to FIG. 2A, a schematic diagram of an example closed-loop system 200, in accordance with the present disclosure, is shown. In some aspects, the closed-loop system 200 may be used for the rehabilitation or treatment of a medical condition of a patient, such as TBI, as well as for other applications. As shown in FIG. 2A, the closed-loop system 200 may generally include a stimulation system 202 coupled to the patient and configured to deliver electrical stimulations to the patient, and a capture system 204 in communication with the stimulation system 202 and configured for gathering and evaluating patient performance, for instance, during a selected task. Specifically, the capture system 204 may generally be configured to receive and process patient input or feedback, and provide triggers or command signals to the stimulation system 202 via wired or wireless connection(s). The stimulation system 202 may then receive the triggers or commands, and deliver selective and time-specific electrical stimulations based on patient input. As described, in some aspects, such stimulations may be applied to more than one brain tissues or brain regions, and in specific relative timing.

By way of a non-limiting example, FIG. 2B illustrates one embodiment of the closed-loop system 200 described above, including a stimulation system 202 and a capture system 204. As shown, the stimulation system 202 may include an implantable device 206 for delivering electrical stimulation to the patient, and a wearable device 208 in communication with the implantable device 206 and configured to control the implantable device 206 using a wired or wireless connection. In some implementations, the implantable device 206 may be fully or partially positioned within the anatomy of the patient, while the wearable device 208 is external to the patient. In other designs, rather than external to the patient, the wearable device 208 may be positioned fully or partially within patient. In yet other designs, the implantable device 206 and wearable device 208 or portions thereof may be combined into a single device. When the wearable device 208 is external to a patient, it may be preferable, although not necessary, that the wearable device 208 communicate wirelessly with implantable device 206.

As mentioned, the implantable device 206 may be configured to deliver therapeutic or rehabilitative electrical signals to various locations or regions of the brain at specific time points, as well as receive signals therefrom. As such, the implantable device 206 may include multiple implantable components (not shown in FIG. 2B), such as electrodes, or feedthroughs fitted with electrical contacts, stimulators, sensors, and other elements. In addition, the implantable device 206 may also include one or more control modules, and other hardware (not shown), for controlling the implantable components. The implantable components may be designed to be positioned about or coupled to specific structures or regions of the patient's brain, either temporarily or permanently. For example, components of the implantable device 206 may configured for placement into or proximate to the caudate, nucleus accumbens, hippocampus, striatum, nucleus basalis, mammillary bodies, subthalamic nucleus or midbrain, as well as other structures. In some aspects, the implantable device 206 may be configured to deliver electrical stimulation to multiple brain regions or tissues, such as the Cd and NAcc, using appropriate stimulations and in specific relative timing.

In some modes of operation of the closed-loop system 200, such as during the rehabilitation of a patient, various behavior or motor tasks may be provided to the patient by the capture system 204 by using audio or visual instructions, or cues. For example, tasks can include tracking a target displayed on a screen, identifying an object physically or verbally, touching a particular region of a touch screen, identifying an object verbally, using a computer mouse, manipulating objects, and so forth. Although such tasks need not require direct patient feedback, in some implementations, patient feedback would be preferable. Also, in some aspects, tasks requiring patient ambulation may be prompted using additional interface devices or systems. Generally, a task may be selected or adapted by the capture system 204 based upon a patient's condition or feedback, or based on input from a supervising clinician. In some aspects, during or upon completion of a given task, the capture system 204 can provide commands or triggers to the stimulation system 202 to stimulate the patient's brain in order to enable or enhance performance of the current task, or a future task.

The capture system 204 may be in general any computing device, apparatus or system configured for carrying out instructions in accordance with aspects of the present disclosure, including capturing feedback from a patient as well as controlling electrical stimulation provided to the patient, via triggers to the stimulation system 202. In some aspects, the capture system 204 may operate as part of, or in collaboration, with a computer, system, device, machine, mainframe, or server. In this regard, the capture system 204 may be a system that is designed to integrate with a variety of software and hardware capabilities and functionalities, and may be capable of operating autonomously. As shown in the example of FIG. 2B, the capture system 204 may be a personal computer, or a workstation, configured with one or more input 210 and output 212 elements. For example, the input 210 can be a keyboard, mouse, joystick, touch screen, or other user interface or input device, while the output 214 can be a visual display, screen, speakers, or other user output device. In other implementations, the capture system 204 may be a portable device, such as a laptop, tablet, smartphone, personal digital assistant (“PDA”), or other mobile or portable device or apparatus. In addition, the capture system 204 may also be in the form of, or include, various wearable elements, sensors, or components capable of the above-described functionalities.

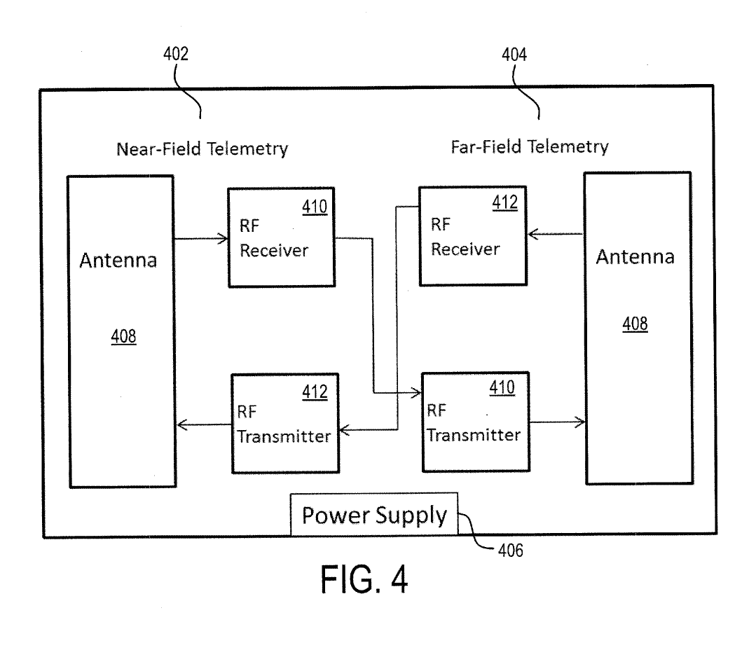

As described, the capture system 204 may be configured provide triggers or command signals to the stimulation system 202 to control delivery of electrical stimulations to a patient. In addition, the capture system 204 may also receive data or information from the stimulation system 202. In some aspects, communication between the stimulation system 202 and the capture system 204 may be achieved wirelessly via the wearable device 208 located proximate and external to the patient. In alternative implementations, communication between the stimulation system 202 and the capture system 204 may be achieved directly with the implantable device 206, in which case the wearable device 208 might be used for checking, programming, or reading out data from the implantable device 206. As such, the wearable device 208, or implantable device 206, may be configured with near-field telemetry capabilities, for communicating signals at a close range, as well as far-field telemetry capabilities, for communicating with the capture system 204 at a far range, respectively. For example, referring specifically to FIG. 4, the respective device may include a near-field module 402, a far-field module 404, and a power supply 406, as well as other components. Each telemetry module, may include an antenna 408, an RF transmitter 410 and an RF receiver 412, coupled as shown. In some designs, the device may also include capabilities for modifying or processing the received signals. By way of example, when far-field communication is not transcutaneous, it may be accomplished using a number of techniques, such as Bluetooth, or other wireless communication protocol.

As described, the wearable device 208 may be placed near the implantable device 206, say at a distance between 2 and 3 cm. Larger or smaller separations between the implantable device 206 and wearable device 208 may also be possible, with telemetry capabilities adapted accordingly. In this manner, communication and power signals can be transmitted to the implantable device 206 via the antenna 408 of the near-field telemetry module 402. In some implementations, the power signals can exist within the transmitted signals, allowing data telemetry and power transmission to occur simultaneously. By way of example, transmitted signals can be in a MHz frequency range, although other ranges may be possible. As such, the transmitted signals may include a variety of information including operational parameters and triggers for generating electrical stimulations using electrical contacts placed at various locations about a patient's brain. In some aspects, communication with the implantable device 206 via the external wearable device 208 can also be used to check the impedance of each contact for assessment of contact longevity and contact breaks. In some aspects, telemetry with the control module of the implantable device 206 will not only allow programmability but also readout capabilities of information stored in the implanted device 206. For example, this information can include existing or previous stimulation parameter settings for each electrical contact, a number of activation events, stimulation times, battery life, and so forth.

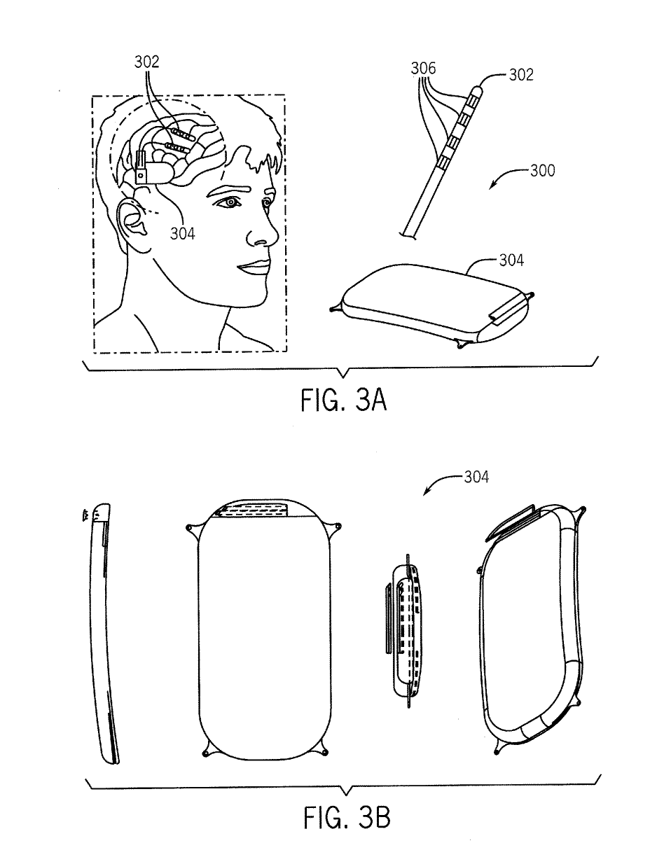

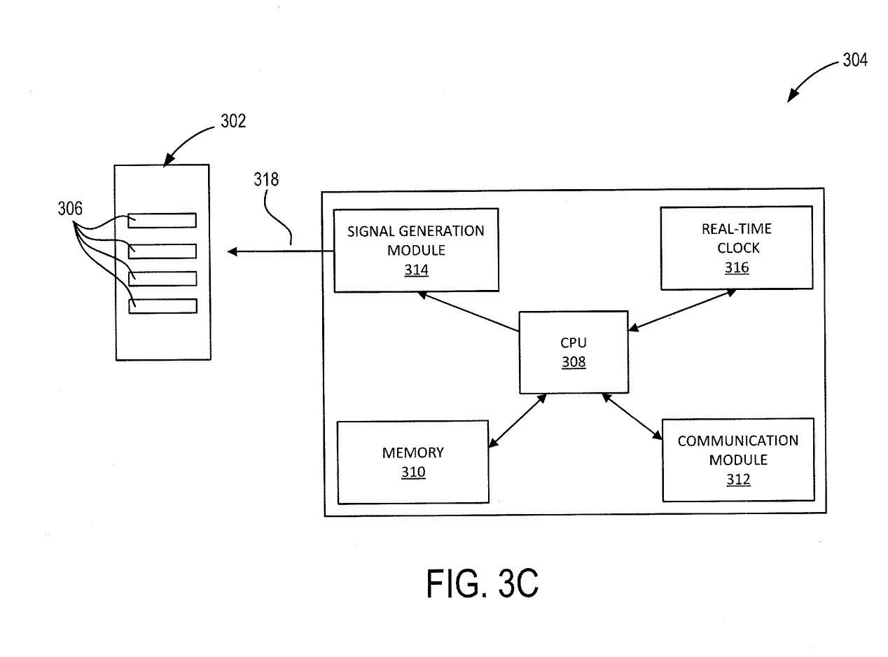

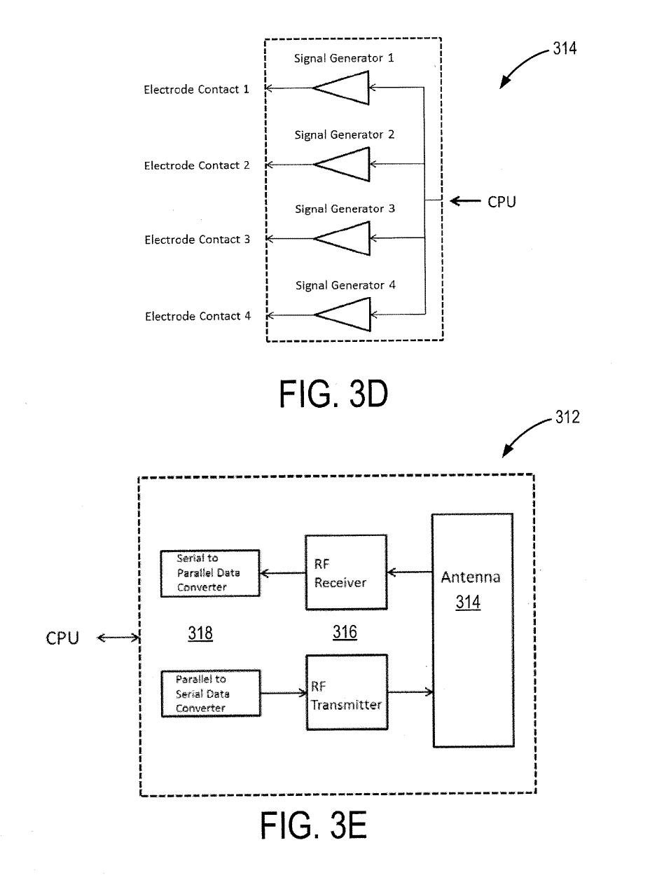

Referring now specifically to FIG. 3A, a non-limiting example of an implantable stimulation device 300, in accordance with aspects of the present disclosure, is illustrated. The stimulation device 300 can include one or more electrodes, or feedthroughs 302 fitted with a number of electrical contacts 306, or stimulators. By way of example, each feedthrough 302 can include 4 electrical contacts 306, as shown in FIG. 3A, although it may readily understood that fewer or more contacts are possible. In some implementations, multiple feedthroughs 302 may be preferable in order to access and stimulate different brain regions or tissues, such as the NAcc and Cd. Optionally, the implantable device 300 may also include one or more sensors (not shown), either integrated into the feedthroughs 302, or configured separately, for monitoring various activities associated with the patient's brain. For example, the implantable device 300 may incorporate one or more recording electrodes or electrical contacts for monitoring electrical and other signals generated in various brain structures. Example signals may include alpha, beta, theta, or gamma oscillations from one or more brain structures, as well as single neuronal firing, or signals associated with various neurotransmitters, such as dopamine, glutamine, or serotonin. Alternatively, the patient's brain could be monitored using one or more of a scalp electroencephalogram (“EEG”) or cortical EEG (not shown).

As shown in FIGS. 3A and 3B, the stimulation device 300 may also include a control module 304 in communication with the electrical contacts 306 or sensors assembled on the feedthroughs 302. In some aspects, the control module 304 is placed subcutaneously on a patient's skull. The control module 304 may also configured to receive triggers and signals for providing electrical stimulation, for example, communicated by a capture system, as described with reference to FIGS. 2A and 2B. During operation, the control module 304 may be configured control, either individually or as a group, the electrical contacts 306 fitted on the feedthroughs 302 to deliver various electrical stimulations spanning a wide range operational parameters. For instance, electrical stimulations may be pulsed, continuous, or intermittent in the form of currents or voltages having various amplitudes, frequencies, periods, waveforms, durations, phases, polarities, and so on. In some aspects, pulsed electrical stimulations may include a number of monophasic and/or biphasic pulses. For example, pulses may be defined by current amplitudes in a range between 0 and 10 milli-Amperes (“mA”), voltage amplitudes in a range between 0 and 10 Volts (“V”), frequencies in a range between 0 and 300 Hertz (“Hz”), and pulse widths in a range between 0 and 250 microseconds (“μsec”). In addition, a series of pulses defining an electrical stimulation may have a duration lasting between 0 to 10 seconds. Electrical stimulations are not limited to the examples above, however, and may indeed include other parameter values. In some aspects, the operational parameters may be modified based upon a patient feedback or performance, and/or brain region or tissue being stimulated.

As shown diagrammatically in the non-limiting example of FIG. 3B, the control module 304 may be rectangular, sized to dimensions approximately 51×25×3 mm³, and encased in a metallic shell, although it may be appreciated that various implementations including sizes, shapes, designs, materials and configurations are also possible. For instance, in some aspects, the control module 304 may be fashioned and dimensioned in a manner appropriate for partial or complete implantation, as well as for operating in accordance with the present disclosure.

The general components of the control module 304 are shown in FIG. 3C, and may include a central processor unit (“CPU”) 308 for controlling the control module 304, a memory 310, such as a flash memory, a communication module 312, a signal generation module 314, a real-time clock 316, and a power source (not shown). As shown, the control module 304 may also include connections, or terminals 318 for transmitting electrical signals, generated by the signal generation module 314, to targeted brain regions or tissues via electrical contacts 306 fitted on the feedthroughs 302.

Specifically, the CPU 308 can be configured to perform a variety functions for controlling the control module 304 using instructions stored in memory 312. In some implementations, the CPU 308 may control the sending and receiving of instructions and operational parameters (for example, via a wireless transcutaneous link in the communication module 312), the storage of the operational parameters and instructions in memory 310, the transmission of the operational parameters to signal generators in the signal generation module 314, the selective triggering of the signal generators to provide electrical stimulations to various brain tissues of a patient, as well as synchronizing various functions using the real-time clock 316. For instance, the CPU 308 may communicate with the real-time clock 316 to determine the timing and synchronization of various electrical stimulations. The CPU 308 may also communicate with the real-time clock 316, as well as other hardware and digital logic circuitry, to accurately store activation times in memory 310 and provide activation counts. By way of example, the CPU 308 can be a programmable microprocessor or microcomputer, which may be custom made or obtained from various computer chip manufacturers.

The signal generation module 314, in communication with the CPU 308, may include a number of signal generators for providing electrical signals to the electrical contacts 306 assembled on the feedthroughs 302. In some implementations, as shown in FIG. 3D, each of the electrical contacts 306 may be individually controlled using separate signal generators. The signal generators can be independently operated, either sequentially or concomitantly, to provide stimulation signals defined by various amplitudes, frequencies, phases, pulse-widths, durations and waveforms, as directed by the CPU 308. In some accordance with some aspects of the disclosure, the signal generators may be controlled to provide electrical stimulations at multiple time points and brain locations. For instance, a first electrical stimulation may be provided to a first location in the patient's brain, such as the NAcc, at a first time point while the patient is performing a task. After or during acquisition of feedback from the patient, a second electrical stimulation may then be provided to a second location in the patient's brain, such as the Cd, where the second electrical stimulation occurs at a second time point relative to the first time point, in accordance with the acquired feedback. In some aspects, the signal generation module 314 may include an output sensing circuit to monitor electrode output (not shown in FIG. 3D), as well as other fail-safe mechanism. This may be desirable, for instance, in order to mediate timed switching for biphasic pulsing

Referring again to FIG. 3C, the communication module 312, in communication with the CPU 308, may be configured to send and receive various signals, as well as receive power. As shown in the example of FIG. 3E, the communication module 312 may include an antenna 314, or an input-output wire coil, an RF receiver and transmitter 316, data convertors 318, as well as other hardware components. In some implementations, the antenna may be configured for transcutaneous wireless two-way communication with an external wearable device, sending and receiving signals when the external wearable device is placed in close proximity. The communication signals may be transmitted through magnetic induction and include information for operating and/or programming the CPU 308. For instance, the communication signals may include triggers or command signals for generating electrical stimulations. In some aspects, transmitted signals may also be configured to power or recharge battery components powering the control module 304. As shown in FIG. 3E, the antenna 314 may be connected to an RF receiver and transmitter 316, which in turn may be connected to serial-to-parallel and parallel-to-serial data convertors 318, respectively. Any information sent or received, as described, may then be processed by the CPU 308.

As mentioned, the control module 304 may be powered by an internal and/or external power source (not shown in FIG. 3C). For example, an internal source may include a standard rechargeable battery, comparable to batteries used in implantable devices (i.e., pacemakers). Alternatively, or additionally, the internal power source may include a capacitor in combination with a regulator, such as a single ended primary inductor converter or dc-dc converter, that together can generate a constant current or voltage output for short periods of time. In some aspects, the capacitor may be charged by an external wearable device, as described with reference FIG. 2B. As such, the control module 304 may include an induction coil, or thin, tightly wound wire that allows for radio frequency (“RF”) telemetry and/or battery recharge by an external wearable device, configured either as part of the communication module 312, or as separate hardware.

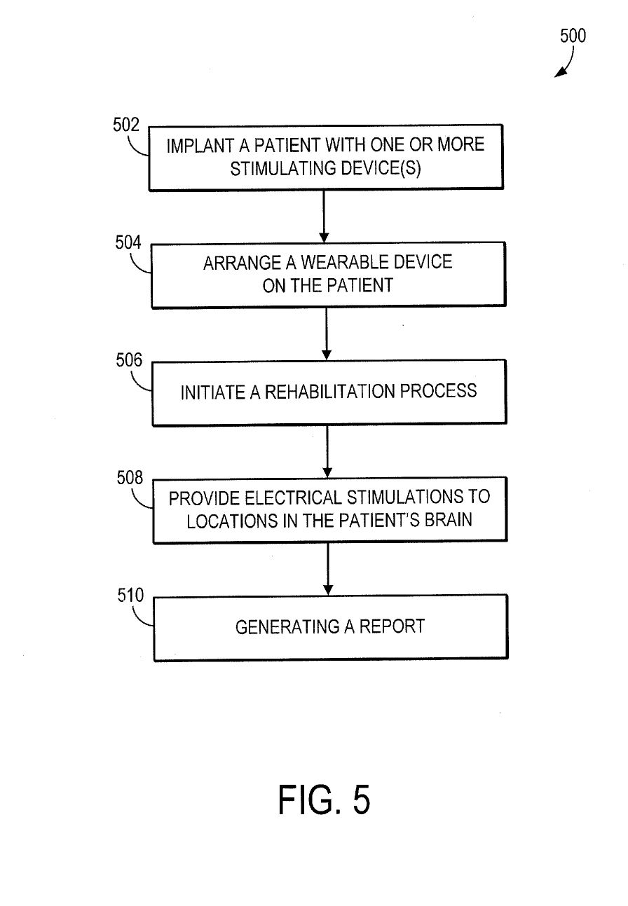

Turning now to FIG. 5, steps of a process 500 in accordance with aspects of the present disclosure are shown. The process 500 may begin at process block 502 with a patient being implanted with an implantable device configured for delivering electrical stimulation to one or more locations in the patient's brain. For example, the implantable device or components thereof may be placed proximate to, or within, locations associated with a hippocampus, a nucleus basalis, a mammillary body, a caudate, a nucleus accumbens, and other tissues, as well as a combination thereof.

At process block 504, a provided wearable device may then be arranged on the patient. As described, such wearable device may be advantageously arranged in proximity to the implantable device and include capabilities for controlling the implantable device, communicating with a capture system for recording, processing, and transmitting data associated with patient performance. In some aspects, following post-implant procedures, stimulation parameters may be set. As described, electrical stimulations may be pulsed, continuous, or intermittent in the form of currents or voltages having various amplitudes, frequencies, periods, waveforms, durations, phases, polarities, and so on. For example, monophasic or biphasic pulses may be defined by current amplitudes in a range between 0 and 10 milli-Amperes (“mA”), voltage amplitudes in a range between 0 and 10 Volts (“V”), frequencies in a range between 0 and 300 Hertz (“Hz”), and pulse widths in a range between 0 and 250 microseconds (“μsec”). In addition, electrical stimulation may have a duration lasting between 0 to 10 seconds. Other stimulation values may be possible.

At process block 506 a patient rehabilitation process is initiated. As described, this may include providing instructions to the patient for performing learning, motor, or other tasks, via the output of a performance capture system, for example. In some aspects, tasks may be tailored to the particular patient's condition, deficit or current progress. At process block 508, electrical stimulation may be provided to various locations in patient's brain via the implantable devices. In some aspects, different locations, such as locations associated Nacc and the Cd of the brain, may be stimulated in a relative timing in order to achieve a target or enhanced performance. Specifically, such electrical stimulation may be modified or adapted at process block 508 based on patient feedback acquired and processed by a capture system. Then, at process block 510, a report of any shape or form, may be generated and provided, for example via a display. In some aspects, the report may include information related to the patient's performance to an assigned task, task completions, as well as other feedback provided by the patient. The report may also include information regarding delivered electrical stimulations, as well as provide a comparison to a baseline or reference performance, or tracking a progress in time.

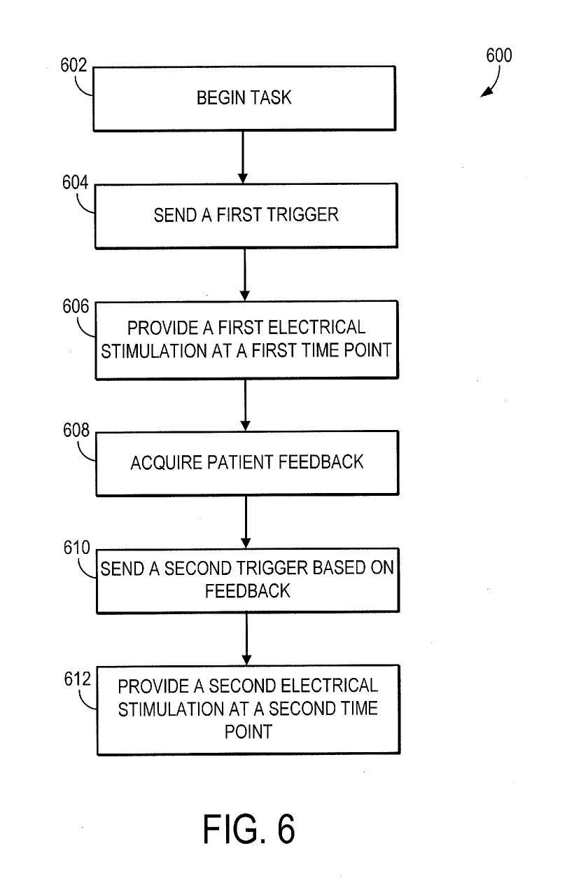

Referring now to FIG. 6, steps of a process 600 for treating or rehabilitating a patient, in accordance with aspects of the present disclosure, are shown. The process 600 may begin at process block 602, where a learning or rehabilitative task may begin. In some aspects, a visual, audio, or other cue, may be provided to the patient to indicate a start of the task. Then, at process block 604, a performance capture system, for example, as described with reference to FIG. 2A, may send a first trigger to a stimulation system to provide an electrical stimulation to selected tissues or region in the patient's brain via selected electrode contact(s). By way of example, the first trigger, and subsequent triggers, can be TTL triggers. In some aspects, the first trigger may be in the form of one or more transmitted signals and include information regarding the timing, duration, and nature of a provided electrical stimulation, as well as specific locations targeted in the patient's brain.

As described, the first trigger may be sent to command the control module of an implantable device to deliver a first electrical stimulation at a first time point, as indicated by process block 606. In some aspects, the first stimulation may be directed to a first location in the patient's brain, such as the NAcc, Cd, or other location. The first stimulation may be described by a broad range of operational parameters including various amplitudes, frequencies, periods, waveforms, durations, phases, polarities, and so on. For example, monophasic or biphasic pulses describing the first stimulation may be defined by current amplitudes in a range between 0 and 10 milli-Amperes (“mA”), voltage amplitudes in a range between 0 and 10 Volts (“V”), frequencies in a range between 0 and 200 Hertz (“Hz”), and pulse widths in a range between 0 and 250 microseconds (“μsec”). In addition, the first stimulation may have a duration lasting between 0 to 10 seconds. It may be appreciated that other operational parameters may be possible.

Following the first stimulation, the patient may be provided with additional instructions or cues associated with the task, and feedback may then be acquired, as indicated at process block 608. Specifically, feedback may be acquired and processed using a capture system, for example, as described with reference to FIGS. 2A and 2B, and include patient responses to the task. In some aspects, feedback provided by the patient may be reported, for example, by way of a display, audio signals, and so on. Following feedback from the patient, a second trigger may then be sent, as indicated by process block 610, to command a second electrical stimulation. Alternatively, the second trigger may be sent during patient feedback. In some aspects, processed feedback from the patient may determine the nature of the second trigger, for example, its temporal occurrence following the first trigger, as well as information associated with second stimulation, such as duration, frequency, current, voltage, pulse width, and so forth, which may be similar or different from the first stimulation.

Following the second trigger, a second stimulation is then provided at a second time point, as indicated by process block 612. In some aspects, such stimulation is directed to points or regions associated with a second location in the patient's brain, such as the NAcc or Cd. In the steps described above, preferably, the latency between a trigger and electrical stimulation, that is to say, the time elapsed between the end of trigger and actual onset of the stimulation, is envisioned to occur within 100 milliseconds, although other timing values may be possible. In some aspects, feedback may also be acquired from the patient following the second electrical stimulation at process block 612, and analyzed to determine and report a performance of the patient.

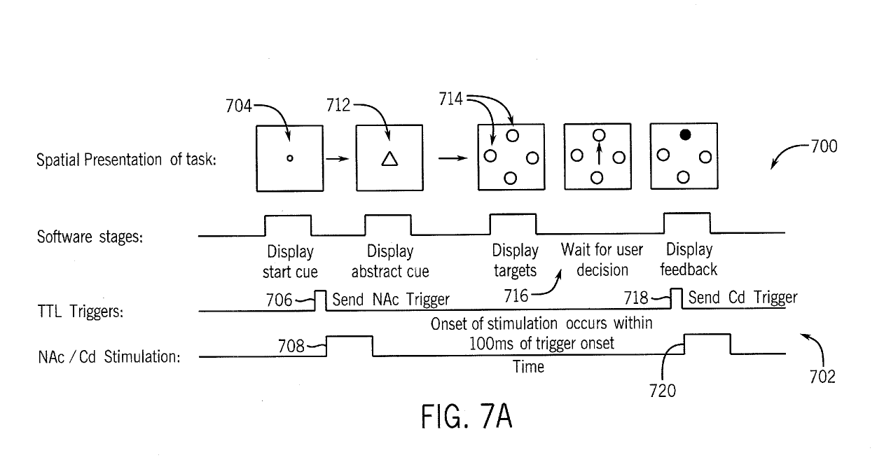

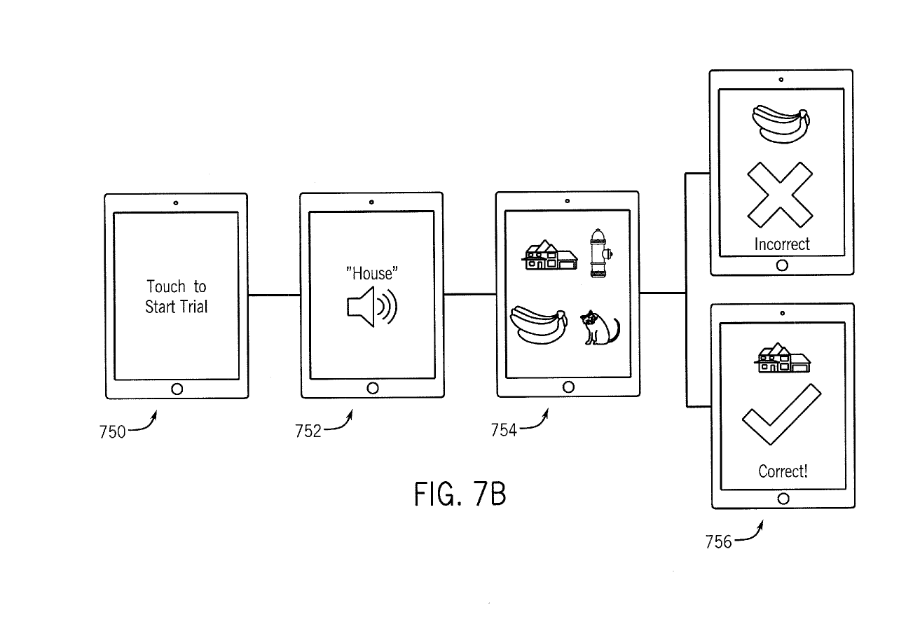

By way of example, the timing sequence for a task-based treatment or rehabilitation process involving electrical stimulation is shown in FIG. 7. In this example, a patient is subjected to a behavioral learning task that includes multiple visual stimuli 700 in combination with multiple electrical stimulations 702, in order to enhance association formations in the brain. However, any task where the patient may learn to make associations through trial and error may be utilized. As seen in FIG. 7, the electrical stimulations 702 may be provided at different time points, each stimulation being in a timed association with respect to the visual stimuli 700 provided, and directed at different locations in the patient's brain. Particularly, after a start cue 704, in the form of a point displayed to the patient, a first trigger 706 is sent. Following a latency, which preferably is less than 100 milliseconds, a first stimulation 708 is then provided to a first location in the patient's brain. As shown in FIG. 7, the first trigger 706 may initiate a first electrical stimulation 708 to the NAcc, although it may appreciate that other points or regions in the patient's brain may also be stimulated. In addition, as described, various electrical contacts or stimulators configured in an implantable device and appropriately selected operational parameters may be utilized to provide the first stimulation 708.

Following the first stimulation 708, the patient is provided with additional visual stimuli 700 over a period time leading up to a second trigger 710, the visual stimuli 700 being in the form of a displayed abstract cue 712 and multiple targets 714. As shown in FIG. 7, the abstract cue 712 includes a triangular shape, while the targets 714 are circular shapes. It may be appreciated that other cues, targets, or other stimuli may be also be provided, depending upon the treatment or rehabilitation process being performed. As indicated by label 710, a decision period 716 is included in the period time, during which feedback is acquired from the patient. Such feedback acquisition would be based on the timing and type of stimuli, as well as the input provided by the patient. In the example of FIG. 7, the patient provides a selection of one of the circular targets 714 based upon the abstract cue 712.

Following the decision period 716, a second trigger 718 is then sent, commanding stimulation of a second location in the patient's brain, in this case, the Cd. A the second stimulation 720 would subsequently follow, preferably in less than 100 milliseconds. In some aspects, provided feedback, and other information, may be displayed during the second stimulation 720, and also feedback acquisition may continue. As illustrated in FIG. 7, the first stimulation 708 to the NAcc begins at a first time point, toward the beginning of the treatment process, while the second stimulation 720 to the Cd begins at a second time point toward the end of the treatment process. However, it may be appreciated that the order, timing and nature of each stimulation may be modified, for instance, based on what would be most beneficial for the condition and performance capabilities of the patient. Specifically, in some implementations, the nature of the second stimulation 720 to the Cd may be adapted based on a patient's condition, the feedback provided, and other variables or conditions. For example, the second stimulation 720 may be modified according to the period time elapsed from the first stimulation 708, the duration of the decision time 716, the selection, input or decision by the patient during the decision time 716, or other time, and so forth.

Referring now to FIG. 7B, another example of task-based rehabilitation process, implemented on a tablet, is shown. It may be appreciated that other implementations may be possible, including implementations on smartphones, laptops, computers, workstations, and so forth. In some applications, the rehabilitation process may be applicable to memory and speech recognition, as well as other applications. As shown, the process may start at step 750 with a visual, or audio, cue, or instruction. As shown, such cue can direct a user to engage the touch screen of the tablet to begin the process. It is envisioned, however, that such cue may involve other instructions, directing the user to perform other movements or actions. For example, a patient may be directed to wave a hand over a camera, to speak, press a button, or perform other activities.

Following step 750, a first visual and/or audio stimulus, indicated at step 752, may then be provided. As shown in the non-limiting example of FIG. 7B, the first stimulus can involve displaying and/or sounding the name of an object. In accordance with aspects of the disclosure, a first electrical stimulation, at specific brain locations, may then be provided to the patient at one or more time points previous to, during, or after, the execution of step 752. Then, at step 754, a group of objects may be displayed, prompting the patient to make a selection consistent with the first stimulus. Then, following the selection, a second stimulation may be provided at step 756, the second stimulation directed to specific brain locations, which may be different from those of the first stimulation, as described, and occurring at one or more time points. As shown in FIG. 7B, in addition to the stimulus, the selection, and accuracy of the selection, be displayed or reported to the patient. It is envisioned that the above-described rehabilitation process may be modified in a number of ways. For example, the task difficulty may be modified, to be easier, or harder, depending upon the condition of the patient, and/or according to directions from a clinician. In addition, more pictures, fewer pictures, similar pictures, different pictures, or visual cues may be utilized.

The approach of the present disclosure was demonstrated to be effective in enhancing the performance of individuals with brain injuries. In one example, learning performance for animal subjects provided with different electrical stimulations, in comparison to no treatment, are shown. In particular, FIG. 8A shows learning curves conveyed as percent correct across trials from one animal. Traces represent a moving average (window size=4) of the correct and incorrect choices made by the animal for each block condition. The curves include A No Stim block (black trace) composed of n=43 blocks (animal 2: n=48 blocks), a NAcc Stim block (blue trace) composed of n=42 blocks (animal 2: n=25 blocks), a Cd Stim block (green trace) composed of n=39 session (animal 2: n=27 blocks), and a NAcc plus Cd Stim block (red trace) composed of n=34 blocks (animal 2: n=20 blocks). Familiar images from all block conditions (gray trace) composed of n=158 blocks (animal 2: n=120 blocks). The inset to FIG. 8A shows the mean percent correct for each of the first three trials (no sliding window). FIG. 8B shows a state-space approach learning curves for each block condition from one animal. Thick areas along traces indicate trials where performance on stimulated trials was significantly different from performance on non-stimulated trials. FIG. 8C shows the distribution of learning criteria for each block condition (top) and final performance for each block condition (bottom). FIG. 8D shows a scatter plot of reaction time sorted by final performance of each block condition and familiar images. The thick black circles represent the mean for each distribution. The dashed line represents a linear regression fit to the mean reaction time for each distribution. The above results demonstrate the advantages of electrical stimulation for enhanced learning, as well as indicate superior results for stimulation multiple brain tissues, in appropriate relative timing, not expected or achievable using individual tissue stimulations.

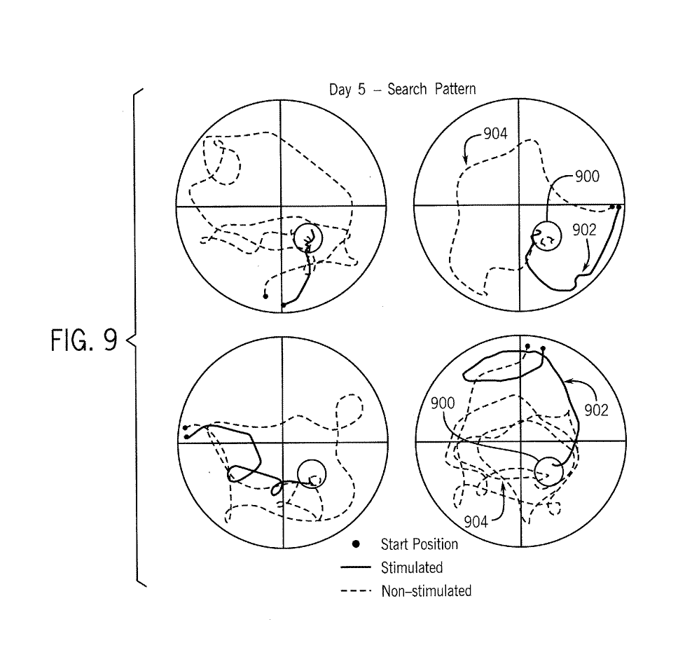

As another example, following TBI, animal subjects were implanted with a stimulation device that targeted the NAcc and the Cd portions of the brain. The animals then performed a behavioral learning task called the Morris Water Maze. The water maze consisted of a small pool that contained a platform to which the patients could swim. The position of the platform was dependent on one of four large abstract visual cues (each separated by 90 degrees) that were displayed on the wall of the pool. The animals were then dropped into the water maze at each of the visual cues once a day for 5 days in order to learn the location of the platform based on each cue. The intervals of the task that triggered stimulation were the start interval, or when an animal was dropped in the water maze, and the reinforcement interval, or when the animal found and rested upon the platform. Two groups of animal subjects were tested. One group received treatment using a closed-loop system, in accordance with the present disclosure, and the other did not. As appreciated from FIG. 9, the animals that received selective and time-specific electrical stimulation, indicated by label 900, performed remarkably better on learning the location of the platform, displaying shorter search patterns from their start positions, as compared to the animals that were not stimulated, indicated by label 902.

As may be appreciated from descriptions above, the present approach has a broad range of applications. For example systems and methods may be used to enhance certain capabilities (e.g. task learning), remedy brain injuries and/or dementia disorders, modify behavior (e.g., by diminishing the effects of depression or motivational problems). As described, a stimulating device implanted into a patient's brain, for example, in locations associated with the caudate, nucleus accumbens, and others, may be configured to deliver appropriately configured electrical stimulations. In one implementation, the stimulating device may be configured to communicate with an external piece of equipment. The patient may be situated in an environment where a learning task may be administered (for example at home or in a clinic). An automated learning process may then be initiated such that when the patient responds appropriately to the given task, the external equipment automatically commands the implanted device to stimulate the targeted area of the patient's brain. In alternative implementations, the stimulating device may be configured to detect certain conditions within the patient's brain and then trigger stimulation when those conditions are detected. One example condition is the presence of theta oscillations occurring with the patient's hippocampus. Other conditions include the presence of alpha, beta, or gamma oscillations in various structures within the patient's brain, detection of a particular or single neuron firing, or the presence of particular levels of neurotransmitters such as dopamine, glutamate, or serotonin. In particular, specific oscillations occurring within the patient's brain (or, in fact, any of these conditions) can indicate that learning is actively occurring with the patient's brain. After detecting the condition, the stimulating device may then deliver stimulating signals to the target area of the patient's brain.

In either implementation, by providing the stimulation during a controlled window of time (e.g., within a period of time or time window occurring shortly after the patient successfully completes a task or a particular brain condition is detected), the patient's ability to learn and perform the task is enhanced. This enhanced learning is useful in a number of circumstances including as a result of brain injuries or various medical conditions. For instance, the present approach may be used during recovery of motor skills or speech. It may be supervised in a semi-automated fashion by a clinician or by a family member, or may be completely automated.

In some aspects, systems and methods described in the present disclosure, can be used to rehabilitate brain disorders by harnessing and augmenting the brain's innate memory circuitry. As described, an implantable device may be positioned completely within the head of a patient, for instance subcutaneous, and accessed via a wireless communications channel. The implantable device may be triggered and recharged by wireless telemetry using an external, wearable device, in the form of a cap, for example. In some aspects, the wearable device may be also configured to communicate wirelessly with a computer. In some implementations, the computer can include software or programming designed for carrying out a broad range of tasks aimed at rehabilitating patients using electrical stimulations. For instance, the computer may programmed to enhance object recognition memory. In that case, stimulations may be delivered based on the patient accurately recalling the name of an object, or recalling the object corresponding to a particular name. In some aspects, a treatment protocol could be adaptive, for instance, beginning with simple objects and gradually progressing to more complex or subtle object target size as accuracy improves. In one implementation, the computer may play pre-recorded words followed by a selection of visual images to enhance speech recognition. In that case, patient selections of the corresponding images would then be coupled with appropriate stimulation. Hence, it may be possible to enhance simple cognitive tasks such as basic mathematics by presenting simple problems and reinforcing selection of correct answers or one can use other simple decision-making tasks. Conversely, it may be possible to attenuate severe anxiety or depression by reinforcing the provocative stimuli with a rewarding stimulus.

By stimulating particular regions of the brain, either in response to external cues (e.g., observations made by a treating physician, or responses detected by a computer), internal cues (e.g., monitoring of particular brain conditions such as oscillations or neurotransmitter levels, as described above), or continuously, a number of brain disorders can be treated. The delivery of stimulating signals in accordance with aspects of this disclosure serves to facilitate the creation of new memories or associations that can be lost as the result of the patient suffering from a particular disease, condition, or trauma. In some aspects, appropriately-timed high-frequency stimulation in the caudate, nucleus accumbens, hippocampus, nucleus basalis, mammillary bodies, and other structures, can enhance memory formation and retention, treat brain injuries, or mitigate conditions such as depression or motivational issues. For instance, treatment within those regions may be of great use in treating patients with stroke, traumatic brain injury, memory disorders such as Alzheimer's Disease, or other conditions. The stimulation may be provided to several different brain areas using intermittent stimulation triggered by the patient's performance on appropriate tasks.

The present disclosure describes systems and methods capable of providing selective electrical stimulation to specific regions of the brain and at specific time points during a behavioral learning task. Specifically, in some implementations, it is envisioned that post-implantation of an implantable device, a patient is provided a wearable device or attachable cap that may be positioned over the implantable device. In some applications, it is envisioned that the cap will be worn during rehabilitative exercises performed using a capture system. As described, such capture system could include a computer, tablet, laptop, smartphone or any device capable of administering or displaying information associated with a behavioral learning task, acquiring input from the patient and transmitting information to the external control module based on the state of the behavioral learning task. Therefore, the patient could fulfill their designated rehabilitation regimen at their own leisure and in a place of their choosing, in an independent or supervised (i.e., clinician or family member) environment depending on the functional state of the patient. For example, a patient could perform rehabilitation at home, performing a behavioral learning task once a day on a tablet while sitting on a couch. It is envisioned that behavioral learning tasks could be customized to the patient's deficits. Accordingly, the capture system would be configured to execute software that runs specific tasks to rehabilitate specific functions. For example, if TBI or stroke has caused a patient to have motor control issues in their arm, the task could entail making coordinated arm movements to touch targets presented on a touch screen tablet placed in front of them.

Therefore, the present approach can be used to enhance patient learning and performance, which can be envisioned to be useful in a number of different circumstances. For example, the present approach could be used to treat deficits in speech recognition by executing responsive stimulation based on the performance of a behavioral learning task that plays a pre-recorded word and causes the patient to select a displayed visual image that is associated with that word. Working under this premise, the present approach could be used with behavioral learning tasks that test object recognition, basic mathematics, fine motor movement and more. Any task that incorporates association formation or conditional learning can be tailored to work with the system and method of the present disclosure. Thus, the present systems and methods may be used to reinforce and enhance motor and cognitive function, thus, improving recovery from deficits. In other envisioned applications, systems and methods described herein may be used for treating mild TBI and stroke patients, specifically within the subacute to chronic phase of recovery, as these phases exhibit motor and/or cognitive deficits that could not be recovered with standard therapy.

It is also conceived that the approach of the present disclosure will provide an advantage to the patient's physician by providing a system that can easily inform on the patient performance and recovery. For instance, patient-specific data, such as a number of triggered activations stored in flash memory of an implanted device, for example, can be readily retrieved and analyzed. In addition, patient feedback acquired using a capture system, can be automatically sent to the physicians' computer over the internet, for example. This allows a physician to track changes in patient performance on any performed task, in real-time. Such performance tracking may help identify new tasks as well as provide information as to whether a treatment modification is warranted.

A novel aspect of the present disclosure includes responsive and selective brain stimulation in correspondence to captured behavioral performance or feedback. Specifically, contrary to previous open-loop methodologies, the present approach utilizes a closed-loop system in which patient input is used to tailor and trigger specific electric stimulations. In addition, the electric stimulations can be intermittent, activated only at specific time points during a behavioral learning task, for instance. As such, the present closed-loop system provides the freedom to selectively activate different regions of the brain at different time points. Hence, the present disclosure provides a means for responsive stimulation that can enhance performance by modulating learning, motivation and memory processes, allowing for neurological disorders to be overcome more quickly and with enhanced effects compared to other approaches.

Another novel aspect of the present disclosure relates to the short duration stimulations, activated at intermittent time points. As described, in some implementations, a stimulating device may provide electrical stimulations as directed by a control module located partially or entirely implanted under a patient's scalp. For short, activated electrical stimulations, such module would not require a large internal source or battery. In addition, such battery could be inductively charged using a wearable device, during the performance of a behavioral learning task, for example. A small battery would allow for a much smaller, less intrusive control module positioned under the scalp without extending over a large surface area or substantial weight. Accordingly, a surgical procedure to implant such device will be substantially simpler compared to the procedures presently employed for current deep brain stimulation devices. Furthermore, electrode wires of currently implanted deep brain stimulation devices extend down the neck patient, and connected to a controller in the chest. This introduces a high risk for bending or breaking of wires as the neck turns, as well as infection in the chest cavity. By contrast, in some implementations, the present approach can circumvent this risk by having the entire closed-loop system located within the cranium.

The above-described systems and methods may be further understood by way of example. The example is offered for illustrative purposes only, and is not intended to limit the scope of the present invention in any way. Indeed various modifications in addition to those shown and described herein will become apparent to those skilled in the art from the foregoing descriptions and the following example, and fall within the scope of the appended claims. For instance, specific electrical stimulation parameters and methods are recited that may be altered or varied based on variables such as amplitude, phase, frequency, timing, waveform, duration, stimulation locations, medical conditions, and so forth.

### Example

Traumatic Brain Injury (“TBI”) is a major public health problem. Current medical and surgical management has improved survival, but not necessarily functional outcome. Hence is a pressing need for effective treatments, particularly in the sub-acute period following TBI. The present study provides evidence demonstrating that precisely timed deep brain stimulation in the nucleus accumbens can be used as an adjuvant strategy to augment neural recovery and enhance behavioral rehabilitation. Through the assessment of behavior, morphology and gene expression in a rodent model of brain injury, it is shown that unilateral brain stimulation in the accumbens can induce widespread cellular adaptation to increase neuronal precursor cells and synaptic density as well as regulate marker genes associated with neural differentiation and migration, cell signaling, and neuroprotection. These results provide a major advance in elucidating the function and mechanisms of brain stimulation as a viable therapy to further the functional recovery of deficits following brain injury.

As described below, the impact of utilizing a closed-loop stimulation strategy, based on precisely activating the NAcc in response to feedback from the animal's behavior can improve particular deficits caused by moderate brain injury. The study was conducted using a rodent model of brain injury evaluated on a visuomotor spatial learning task. Results reveal that brain stimulation was able to induce behavioral change. Specifically, injured animals treated with phasic stimulation in the NAcc during the reinforcement epoch of a task performed significantly better than untreated injured animals. Furthermore, it was found that treated animals were able to return to an uninjured performance baseline and showed long-term memory benefits of learned behaviors from the task. Previous work by the inventors showed that closed-loop striatal stimulation in normal animals improved learning, and that such improvement was mediated, at least in part, by enhancing phasic dopamine release. In this study, it was demonstrated for the first time that closed-loop striatal brain stimulation can enhance recovery in a realistic model of brain injury. Interestingly, it was also found that in addition to the already demonstrated increase in phasic dopamine release, stimulated animals exhibited increased bilateral neuronal proliferation in prefrontal cortex as well as bilateral synaptic density throughout cortical and subcortical regions, revealing widespread gross cellular adaptation induced by modulating the corticostriatal system.

In an effort to identify potential molecular mechanisms underlying the observed behavioral and cellular adaptations, a high-throughput RNA sequencing (RNA-seq) was also performed. Findings revealed pervasive molecular adaptations in numerous loci throughout the brain, supporting the notion that stimulation is able to modulate key genes associated with neural network recovery that can overcome TBI-induced pathology. Taken together these findings demonstrate that closed-loop striatal brain stimulation can be a potent strategy for treating specific impairments following brain injury and that its mechanism of action is much broader than previously considered, including neurogenesis, synaptogenesis and wide-spread changes in gene expression that act in concert to greatly enhance speed and magnitude of functional recovery. Methods of this study are described below.

Controlled Cortical Impact:

Adult (10 weeks old) male C57BL/6 mice were anesthetized with isofluorane and mounted on a stereotactic frame (Kopf Instruments, Tujunga, Calif.). A 10 mm midline linear incision was made over the skull with a 3.5 mm right parietal craniotomy bordering the coronal suture anteriorly, and the sagittal suture medially. The bone flap was then removed. The electromagnetic impactor (Leica Biosystems, Buffalo Grove, Ill.) with 3 mm diameter tip was positioned flush with the dura surface. Injury was induced using impactor velocity of 5.2 m/s, depth of 2.65 mm, and dwell time of 100 milliseconds. After injury, the bone flap was replaced and the incision closed with interrupted absorbable suture and given one week to recover. Control animals underwent anesthesia and craniotomy but without cortical impact. Of all mice that underwent impact, the procedure resulted in one intra-operative death.

Brain Stimulation Electrode Implant:

After recovery from cortical impact all mice underwent electrode placement, implanted with a 3-contact, concentric, miniature deep brain stimulation electrode (Rhodes Medical Instruments, Summerland, Calif.). The electrode was designed with a 0.1 mm distal contact, a second 0.1 mm contact located 1.35 mm proximal along the shaft, and a ground contact located just below a 3-pin connector. Mice were anesthetized as described previously and repositioned in the stereotactic frame. The previous incision was reopened. A 0.2 mm right frontal craniectomy was performed over the implant site. Implant coordinates (from bregma, 1.10 mm anterior, 1.35 mm lateral, 3.82 mm ventral) were chosen to position the distal contact in the nucleus accumbens and the proximal contact in the caudate, with the ground contact resting just below dura. The electrode was cemented in place using acrylic dental cement. Animals were given one week to recover.

Stimulation Parameters:

In the stimulation group, the nucleus accumbens and/or caudate contacts were used as the cathode and a sub-dural contact as the anode. Stimulation was delivered as constant current with symmetric biphasic, cathodic leading square wave pulses. High-frequency stimulation parameters were set to 50 μA, 130 Hz, 160 μs pulse width. For low-frequency stimulation, frequency was changed to 50 Hz. Bursting stimulation used in the real time place preference assay utilized 500 ms trains of the high-frequency stimulation parameters with 500 ms in-between trains.

Morris Water Maze:

Visuo-spatial associative learning was assessed via a Morris water maze setup. A white pool (120 cm diameter, 100 cm deep) was filled with water to 70 cm depth. In the northwest quadrant, a round, clear plexiglass platform 10 cm in diameter was positioned 1 cm below the surface of the water. Each mouse was subjected to four trials per day, once at each of the four starting locations marked with abstract cues (north, south, east, and west) and placed in the pool facing the cue mounted on the wall of the pool. All mice were tethered by their head caps to an overhead wire. Mice were given a maximum of 60 seconds to find the platform. If a mouse failed to reach the platform by 60 seconds, it was placed on the platform by the experimenter and allowed to remain there for 20 seconds. At the conclusion of the 5- or 12-day testing period, a probe trial was done in which mice were placed in the tank with the platform removed and latency in the target quadrant was measured. All behavior (i.e. search path, latency, distance, etc.) was captured using digital video and a custom automated tracking system designed in MATLAB (Mathworks, Natick, Mass.). For the stimulation group, a total of five seconds of stimulation was delivered five seconds after arriving on the platform. In the early nucleus accumbens stimulation group, 5 seconds of stimulation was delivered while the animal was facing the directional cue, prior to release into the water. For retention testing, animals were allowed to rest in their home cages for seven days, then retested without any stimulation delivery for an additional five consecutive days.

Wire-Grip Test:

The wire-grip test was conducted to establish baseline motor function. Animals were placed at the center of an 18-gauge wire suspended taut between two poles 20 cm above the ground and the degree of attachment and movement of the mouse scored. A score of 0 was given if the mouse fell within 30 seconds, 1 point for grasp with a single extremity, 2 points for grasping with multiple extremities, 3 points for grasp with multiple extremities and the tail, 4 points for moving along the wire to the pole, and 5 points for climbing down the pole within 60 seconds. Animals were tested on post-operative days 3, 5, and 7 after CCI, as well as post-operative day 4 after electrode placement. Injured animals were divided into treated (receive stimulation during testing) and untreated (did not receive stimulation during testing) based on their average wire grip scores. The average test score for each animal's was calculated and each group was determined such that the total average, across all animals in each group, was equivalent. This ensured that treated and untreated groups were comprised of animals with equally assessed motor impairment.

Real-Time Place Preference:

To evaluate for a hedonic or aversive response to stimulation in the nucleus accumbens, the real-time place preference assay was utilized. Mice were placed in a 20 cm×20 cm square chamber bisected by a wall with a 3 cm door. They were allowed to range freely to either side of the chamber for 30 minutes with constant or bursting stimulation delivered while they were located on the stimulation-paired side of the chamber. Automated video tracking was used to record time located on each side.

Immunohistochemistry:

Three days after the conclusion of MWM testing animals were injected 50 mg/kg BrdU daily for five consecutive days. Six hours after the final injection animals were anesthetized with isofluorane as previously described and underwent transcardiac perfusion with 10 mL phosphate-buffered saline followed by 10 mL 4% paraformaldehyde. The brains were extracted and green tissue dye was applied to the location where the electrode probe was extracted. Brains were post-fixed in 10% formalin for 48 hours, and were then bisected sagittally and placed into formalin before processing.

Both hemispheres of the fixed and processed brains were embedded in paraffin, medial side down, and were sectioned into 8 μm thick sagittal slices. 12 sections were taken at 6 different levels: the first level at the start of the faced block, the next 150 μm later, then at the level of the green tissue dye, then two more levels spaced at 150 μm apart, and a final level 1000 μm from the previous level. Deparaffinized slides underwent citrate buffer antigen retrieval, and were incubated for 1 hour at room temperature (RT) with the following primary antibodies: rat monoclonal anti-BrdU (1:40; Abcam AB6326), rabbit polyclonal anti-Synapsin (1:100; Abeam AB64581), and rabbit polyclonal anti-NeuN (1:500; Abeam AB104225). Slides were treated with the following biotinylated secondary antibodies for 30 minutes at RT: biotin goat anti-rabbit IgG (1:200; Abeam AB6720), biotin goat anti-rat IgG (1:200; Abeam AB6844). Slides underwent a TSA (Tyramide Signal Amplification) step using a FITC-TSA kit (1:50; Perkin Elmer NEL701A001KT) and a Cy3-TSA kit (1:50; Perkin Elmer NEL704A001KT) for 8 minutes at RT. Slides were mounted with a Dapi counterstain medium (Vectashield).

Iron Deposition Staining:

Mice were chronically implanted with electrodes as described previously. They were stimulated with the high-frequency stimulation parameters for 15 seconds. Mice were immediately perfused following stimulation, electrode implant sites were marked with green tissue dye and brains were processed as described previously. Brains were stained for iron deposition using the following protocol: slides were deparaffinized, immersed in a solution equal parts 20% HCl and 10% potassium ferrocyanide (Sigma-Aldrich) solution for 20 minutes, washed 3 times in distilled water, counterstained with nuclear fast red for 5 minutes, rinsed, dehydrated, and then cover-slipped with a resinous mounting medium. Slices of mouse spleen were used as a positive control.

Imaging and Quantification:

All stained slides were imaged using an upright fluorescent microscope (E800; Nikon), and then captured and analyzed using a camera integrated software (Basic Research; NIS-Elements). Analysis was done blind to the behavioral group and the behavioral results. Cell counting was done on 8 μm thick slices under a 40× oil immersion objective in four regions of interest in each hemisphere: hippocampus, subventricular zone, striatum, and anterior rostral migrating stream. Each animal yielded 5 stained sections of each antibody combination. The total number of Dapi⁺, NeuN⁺, and BrdU⁺ cells were calculated by the automated object count software. Automated counts were verified by hand counted data for one section in each animal, confirming consistency between automated and hand counts. BrdU⁺/NeuN⁺ co-labeled cells were hand-counted based on fluorescence color, and averaged across the 5 sections. For Synapsin intensity labeling, raw intensity histograms were generated for each stained slide and average pixel intensity calculated based on binned intensities by pixel. Negative control slides, produced following identical staining protocol as above without application of the anti-Synapsin primary, showed no difference in background staining between treatment groups. Intensity labeling data graphed represents average pixel intensity across the 5 sections obtained in each hemisphere.

Whole Transcriptome Tissue Preparation:

RNA-seq was used to study changes in transcriptional gene regulation in the setting of TBI and DBS. Three animals in each of three groups, control, untreated and treated underwent controlled cortical injury (or craniotomy without injury for control animals) and electrode implant as previously described. Seven days after electrode implant, treated animals were placed in an 8 cm×20 cm chamber and received stimulation for 60 minutes with the high-frequency parameters described above. Control and untreated mice were placed in the chamber for 60 minutes but did not receive stimulation. Mice were immediately anesthetized and euthanized via cervical dislocation. Brains were extracted expeditiously and placed on an iced 1.0 mm brain slicer matrix (Zivic Instruments, Pittsburgh, Pa.). From 1 mm thick coronal slabs, the right nucleus accumbens, left hippocampus, and left prefrontal cortex were isolated and placed in 1.5 mL RNA-later solution (Qiagen, Valencia, Calif.). After 24 hours at 4° C., samples were transferred to −20° C.

RNA-Sequencing:

Total RNA was isolated from each sample using the RNeasy Plus Kit (Qiagen, Valencia, Calif.) per manufacturer's recommendations. RNA concentration was determined with the NanopDrop 1000 spectrophotometer (NanoDrop, Wilmington, Del.) and RNA quality assessed with the Agilent Bioanalyzer (Agilent, Santa Clara, Calif.). The TruSeq RNA Sample Preparation Kit V2 (Illumina, San Diego, Calif.) was used for next generation sequencing library construction per manufacturer's protocols. Briefly, mRNA was purified from 100 ng total RNA with oligo-dT magnetic beads and fragmented. First-strand cDNA synthesis was performed with random hexamer priming followed by second-strand cDNA synthesis. End repair and 3′ adenylation was then performed on the double stranded cDNA. Illumina adaptors were ligated to both ends of the cDNA, purified by gel electrophoresis and amplified with PCR primers specific to the adaptor sequences to generate amplicons of approximately 200-500 bp in size. The amplified libraries were hybridized to the Illumina single end flow cell and amplified using the cBot (Illumina, San Diego, Calif.) at a concentration of 8 pM per lane. Single end reads of 100 nt were generated for each sample and aligned to the UCSC mm10 mouse genome. Raw reads generated from the Illumina HiSeq2500 sequencer were de-multiplexed using configurebcl2fastq.pl version 1.8.4. Quality filtering and adapter removal was performed using Trimmomatic version 0.32 with the following parameters: “SLIDINGWINDOW:4:20 TRAILING:13 LEADING:13 ILLUMINACLIP: adapters.fasta:2:30:10 MINLEN: 15”. Processed/cleaned reads were then mapped to the UCSC mm10 genome build with SHRiMP version 2.2.3 with the following parameters: “--qv-offset 33 --all-contigs”. Differential expression analysis was performed using Cufflinks version 2.0.2; specifically, cuffdiff2 and usage of the general transfer format (GTF) annotation file for the given reference genome with the following parameters: “--FDR 0.05 -u -b GENOME”. Triplicates of each condition were used for analysis. Cuffdiff2 was used to calculate a p-value for each pair-wise comparison across conditions in each region. The false discovery rate corrected p-value was also reported for each comparison as a q-value. Significance in differential gene expression was set at q<0.1.

Statistical Analysis:

All distributions passed tests for normality (Kolmogorov-Smirnov) and for equal variance (Levene Median), unless noted differently. Non-normal distributions were compared with the Mann-Whitney rank sum test.

### Results

### Brain Stimulation Following Brain Injury Enhanced Behavioral Performance

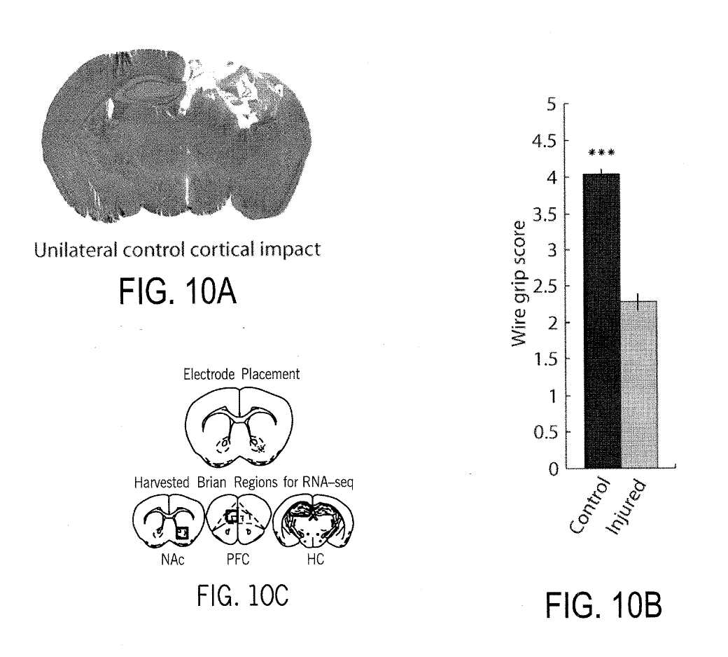

The evaluation of brain stimulation in the NAcc for the purposes of enhancing recovery following TBI was carried out in a rodent model (C57BL/6 mice) using a controlled cortical impact injury, as described. Mice that received a craniotomy with no impact were considered the baseline control, while mice that received a unilateral impact on the dura were considered the TBI test group. The cortical impact site bordered the cranial coronal suture anteriorly and the sagittal suture medially, with a depth of impact that resulted in the complete unilateral destruction of the hippocampus (FIG. 10A). The severity of TBI was classified as moderate and a standard wire grip test (FIG. 10B) was used to assess motor function post-injury, along with cage behavior to assess signs of serious distress and abnormal behavior. Seven days after injury all animals were implanted with a miniature brain stimulation lead, ipsilateral to the injury that terminated in the NAcc core (FIG. 10C).

As described, injured animals were split into two groups, treated and untreated, of which treated animals received stimulation with parameters analogous to clinical biphasic high-frequency brain stimulation (−50 μA, 130 Hz, 80 μs per phase). Behavioral testing was carried out two weeks after injury in a sub-acute phase of recovery, in which the brain is in a state of recovery and injury effects have stabilized, allowing for an evaluation of stimulation as a rehabilitation treatment. The well-validated visual spatial learning protocol of the Morris water maze was utilized to assess the effects of stimulation.

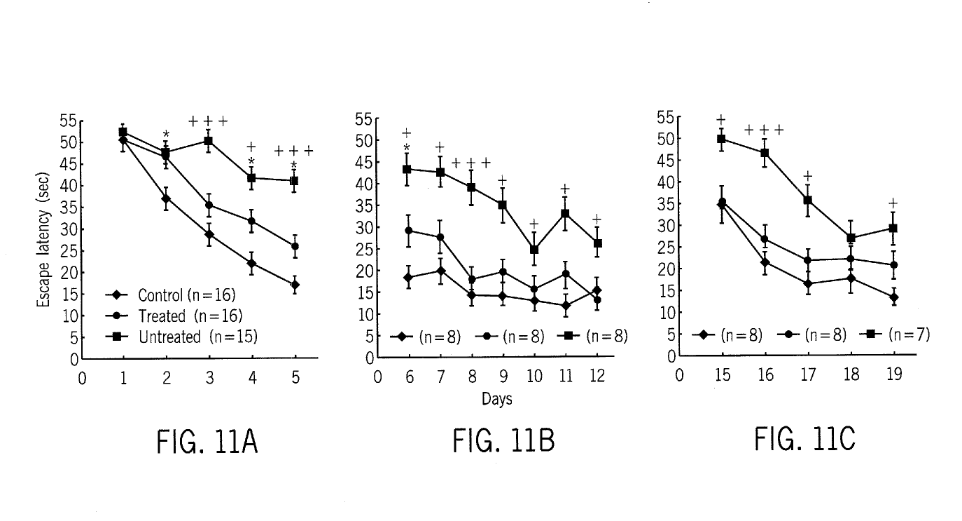

During the task, treated mice received five seconds of stimulation upon reaching and resting on the hidden platform, a strategy intended to reinforce goal location. Learning performance was assessed by mean escape latency. Initially, all animals were tested across five days, in which escape latency decreased in all groups. Injured animals, however, exhibited moderate cognitive deficits on the behavioral task, with significantly longer escape latencies compared to control animals (FIG. 11A). Importantly, injured animals treated with stimulation demonstrated less impairment than untreated animals with a significant enhancement in learning after two days.

Following five days of testing, each group was split, with half of the mice continuing behavioral testing and half receiving a ten day resting period. Continued testing revealed that after day six, escape latency performance of treated animals was not significantly different from control animals, and both groups reached a similar post-training performance plateau (FIG. 11B). Furthermore, the distribution of learning rate coefficients (control: −0.1097, treated: −0.1099, untreated: −0.06) derived from a log-linear regression of mean escape latency across the twelve days of testing revealed that treated mice learned at a much faster rate than untreated mice and approximately to the same extent as control mice. Thus, phasic stimulation in the NAcc during the task was able to enhance the performance of injured animals, which learned at a faster rate and to a greater extent than untreated injured animals. Mice that received a ten day rest period after initial testing were retested on the behavioral task without stimulation and with the same platform location. Day one performance on the task conveyed a significant result, in which untreated injured animals exhibited a total loss of task understanding with performance reverting back to a naïve state, while previously stimulated and control animals showed only moderate loss in performance with a quick rebound to baseline as testing continued (FIG. 11C). There was no significant difference in performance between previously treated mice and control mice, suggesting that the previously applied brain stimulation strategy provided long-term learning and memory benefits.

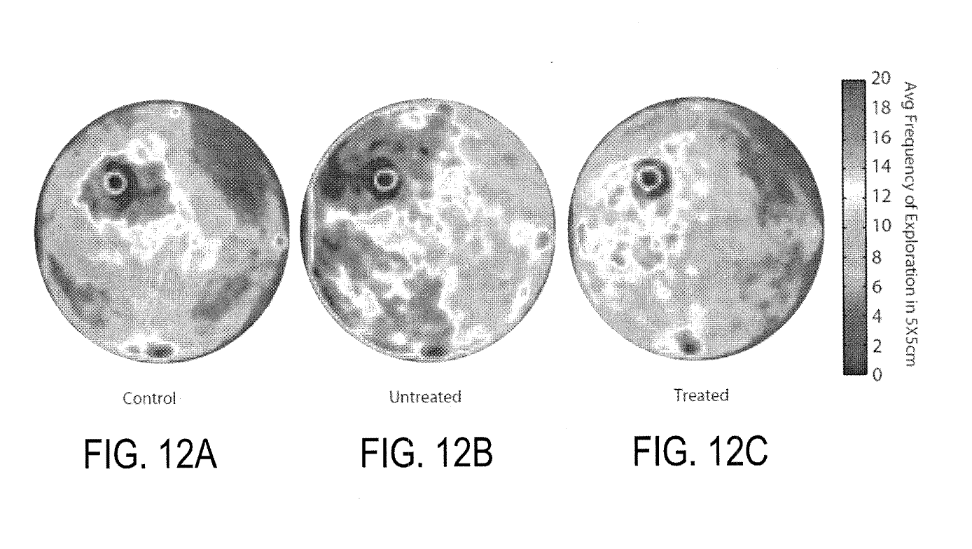

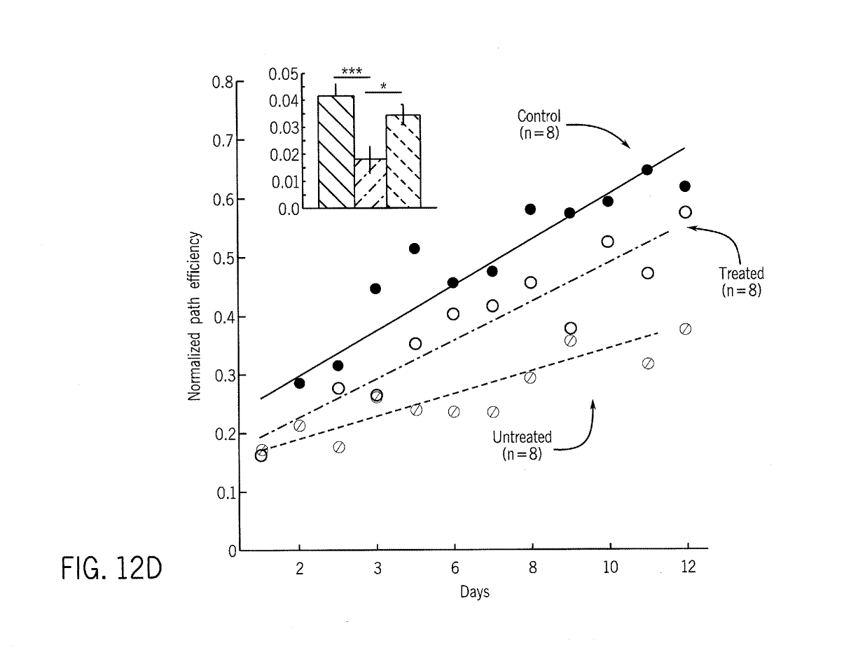

In an effort to understand the trends of escape latency, as there were no differences in average velocity between groups, efficiency in path exploration was evaluated across the twelve days of continuous testing. There was a stark difference between search patterns of control and untreated injured mice. Control animals exhibited a focused search near the platform while untreated animals showed distributed search patterns that encompassed most regions of the maze (FIGS. 12A and 12B). Treated mice demonstrated more distributed search patterns than control animals but targeted regions near the platform (FIG. 12C). Linear regression on the path efficiency for each group (FIG. 12D), calculated from the search patterns of each day, revealed that control animals and treated animals improved across days of testing and at a similar rate (control: 0.038, treated: 0.033). Untreated animals did not show the same rate of improvement (untreated: 0.019) with path efficiency scores and derived rates of improvement that were significantly worse than treated and control animals. These results indicate that the applied brain stimulation strategy allowed treated animals to develop more efficient search strategies.

### Brain Stimulation in the NAcc Did not Induce Hedonic Response

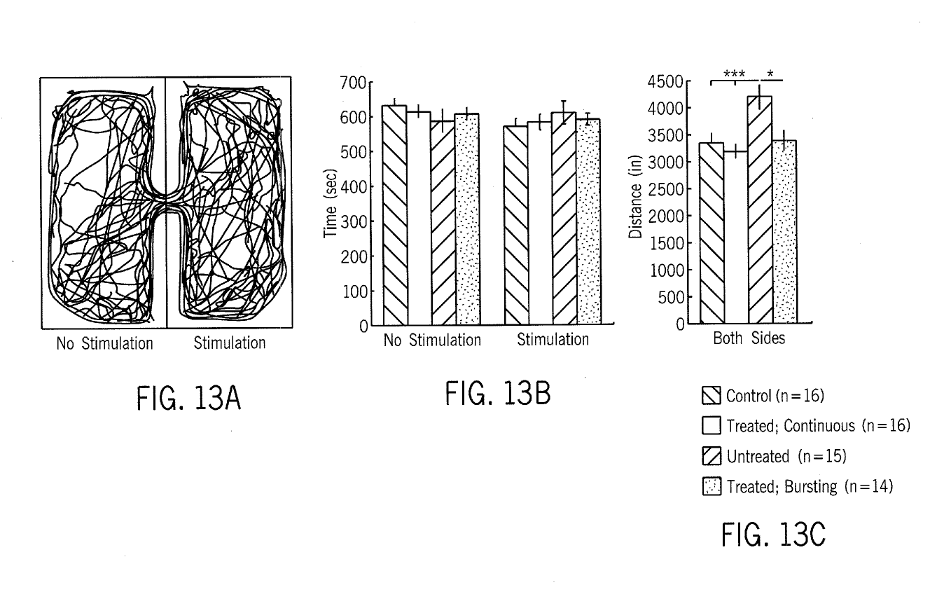

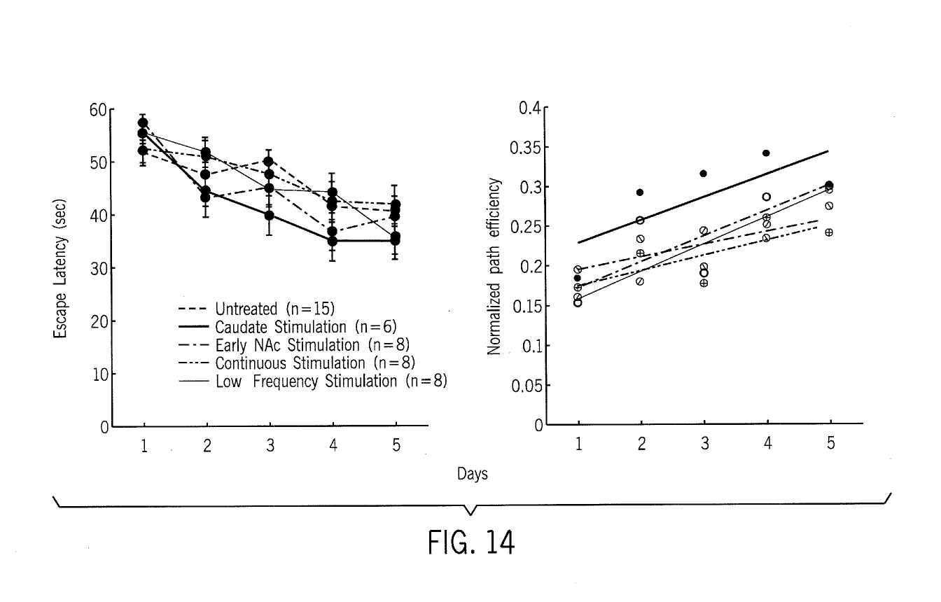

To examine the possible role of stimulation in the NAc as a hedonic stimulus, all animals were tested on a real time place preference task. For treated animals, testing occurred under two different brain stimulation settings, continuous or bursting, on the stimulated-paired side of the environment (FIG. 13A). Regardless of the setting, there were no significant differences between groups in time spent exploring either side of the environment (FIG. 13B). This finding indicates that stimulation did not lead to a simple hedonic response, rather, the brain stimulation strategy acted to enhance reinforcement of goal location. Nevertheless, it was found that untreated injured mice were hyperactive during the task, traveling a significantly greater distance during exploration (FIG. 13C). Aggressive and hyperactive characteristics have been previously reported in brain injured animals. Interestingly, this behavior was not observed in treated mice and no significant difference in the distance traveled on either side of the environment was found when compared to control mice, suggesting a normalization in behavior due to previous brain stimulation treatment.

Several other control experiments were conducted to verify the therapeutic benefit of the applied brain stimulation strategy targeted in the NAcc. Behavioral testing was repeated with new injury groups, including paradigms in which low frequency (50 Hz) was used, stimulation was applied continuously during the task, stimulation was applied at a different temporal epoch of the task during placement in front of a visual cue, and stimulation was applied in different brain region, the Caudate Nucleus (FIG. 4). In each case, it was found that there was no significant difference in behavioral performance between treated and untreated injured mice. These results indicate the importance of targeting appropriate brain structures with induced-activity at relevant learning epochs.

### Promoting Neuronal Precursors and Restoring Synaptic Density

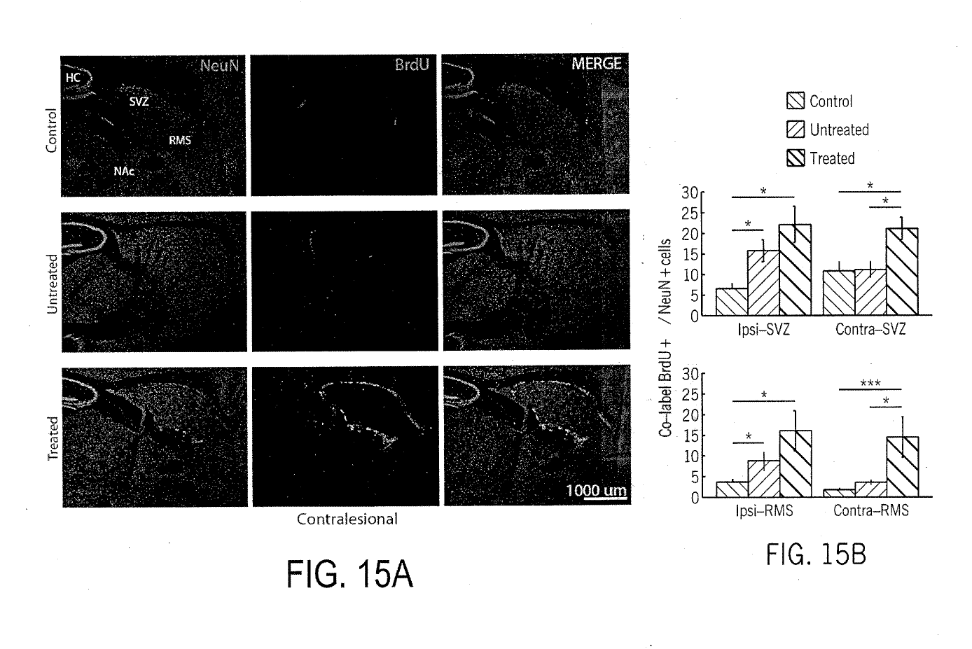

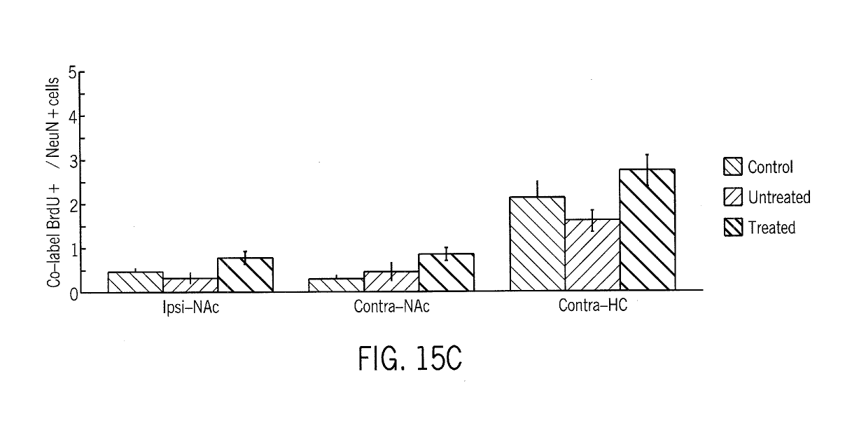

Next, it was investigated whether stimulation in the NAcc generated activity-dependent neurogenesis as a potential underlying mechanism of therapeutic benefit during recovery. As such, mice were injected with the proliferation marker BrdU after completion of behavioral testing. Evaluation of bilateral labeling was focused to key regions of interest including the hippocampus, a major player in spatial memory, the subventricular zone (“SVZ”), a region involved in learning and a known site of rodent neurogenesis, the rostral migratory stream (“RMS”), a migratory route that allows for the relocation of neuronal precursors that originated in SVZ, and the NAcc, a major input node of the basal ganglia integral to learning, memory and motivation. Increased BrdU incorporation was predominantly found in frontal brain regions (FIG. 15A), but was also observed in the sub-cortical structures (FIG. 15C).

Both treated and untreated injured mice showed increased labeling in the ipsilesional SVZ compared to control mice. This was not surprising, as brain injury has been shown to activate neuroregenerative mechanisms. Importantly, increased labeling in treated mice was seen bilaterally, with significantly greater BrdU incorporation than both the untreated and control mice on the contralesional side. These results indicate that unilateral stimulation in the NAcc enhanced the presence of bilateral neural progenitor cells, likely through promotion of neurogenesis or prolonged neuronal survival. Interestingly, it was also found that treated mice showed bilateral increase in BrdU incorporation in the RMS, with a significant increase on the contralesional side when compared to untreated and control mice. This finding suggested that stimulation can accelerate or preserve the migration of newly generated neurons, a potentially important mechanism for augmented recovery following TBI.

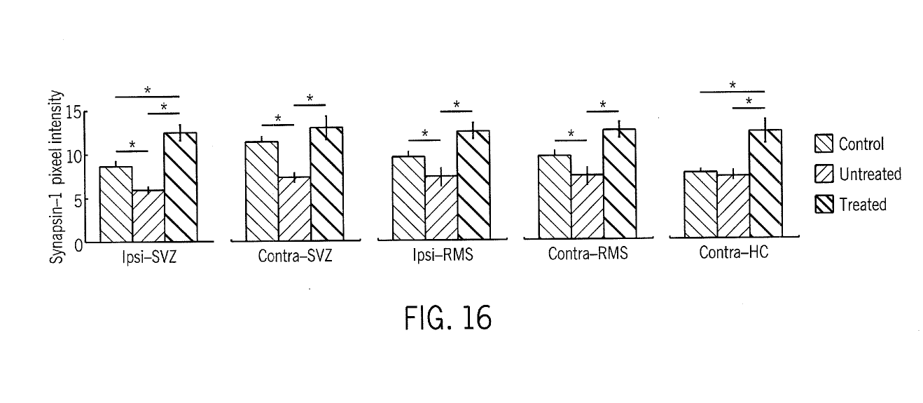

To address the question of whether stimulation-induced activity in the NAcc altered markers for synaptic function, the mean pixel brightness of synapsin-1 labeling was evaluated. Notably, this analysis revealed diminished labeling in untreated injured animals compared with control animals in the NAcc (FIG. 15B), SVZ and RMS (FIG. 15C). A result of this is likely due to a loss of incoming projections from the ablated hippocampus, particularly the subiculum. Imaging of the NAc in untreated mice showed a loss of synapsin-1 density between cell bodies, where presynaptic terminals would converge on dendrites. This trend was not observed in treated animals (FIG. 15B). In contrast, labeling in treated mice was not different from control mice, with the addition of significantly brighter labeling in ipsilesional SVZ and contralesional hippocampus (FIG. 16). When compared to untreated mice, bilateral synapsin-1 labeling was significantly brighter in all regions of interest. Experience-dependent synaptic plasticity in the NAc is believed to be responsible for long-term stabilization of spatial information. These findings indicate that the applied unilateral brain stimulation strategy in the NAcc was able to restore synaptic density bilaterally in injured mice to the level of control mice, providing a mechanism for the enhanced learning and long-term memory benefits observed during behavioral testing.

### Molecular Profiling of Stimulation-Induced Cellular Adaptations

To characterize specific gene expression changes induced by NAcc stimulation in the injured brain a transcriptome-wide differential gene expression analysis was performed. Initial assessment focused on the expression of immediate early genes (“IEGs”) to confirm the molecular response to local stimulation, in accordance with previous studies that have shown increased synaptic activity and IEG expression in the setting of electrical stimulation. Accordingly upregulation of the IEGs Fos (44.1%), Nptx1 (51.3%), Npas4 (56.8%), and Ier2 (79.7%) was found in the NAcc of stimulated animals. No significant change in gene expression was found in or between untreated and control animals. Interestingly, EGR1 (40.0%) and EGR3 (47.4%) were also upregulated in the contralesional hippocampus. These genes have been shown to be induced by transsynaptic activity and play a role in spatial memory consolidation in the hippocampus. These findings suggest a widespread interhemispheric effect of unilateral NAcc stimulation that can alter key genes in regions of learning and memory circuitry.

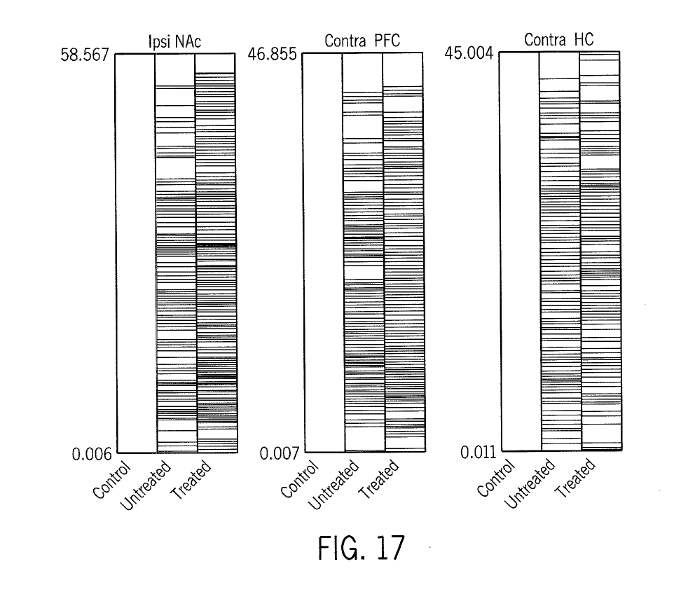

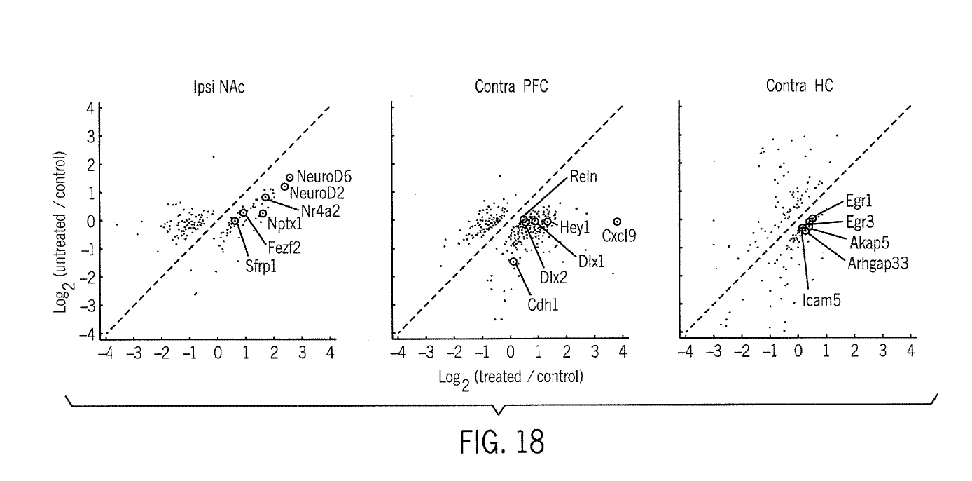

Given the observed labeling of neural progenitor cells and synaptic density, gene expression assessment was focused on three regions of interest, ipsilesional NAcc and contralesional hippocampus and PFC. Direct comparison of expression between control and injured animals, untreated and treated, demonstrated broad shifts in expression (FIG. 17), revealing a gradient for the characterization of differential expression caused by injury and then altered by DBS treatment. Evaluation of the fragment per kilobase of transcript per million mapped (“FPKM”) read values for untreated and treated animals, normalized to values for control animals (FIG. 18), identified a series of significantly differentially regulated genes. Importantly, the functional significance of a subset of identified genes complemented immunohistochemistry (“IHC”) findings, with other gene profiles relating to signal processing, neuroprotection, and neural migration.

### Brain Stimulation in the NAcc Upregulated Transcription of Genes for Synaptic Organization

Expression levels of the transcription factors neurogenic differentiation 2 (“NeuroD2”) and 6 (“NeuroD6”) were upregulated greater than 2-fold in treated versus untreated animals. NeuroD2 and NeuroD6 are basic helix-loop-helix transcription factors associated with synapse regulation and formation. NeuroD2 is a key regulator of cortical-subcortical connections with experiments in knockout mice demonstrating disruption in synapse maturation. Similarly, NeuroD6 is expressed in mature neurons and has an established role in neurite outgrowth as well as regeneration. Given the role of these neurogenic differentiation factors in synapse formation, NeuroD2 and Neurod6 appear to be important regulators of activity-dependent synapse development. The significant upswing in expression of these genes due to stimulation provides a molecular basis for the restoration of cortical and subcortical synaptic density observed via IHC analysis.

### Brain Stimulation Leads to Increased Expression of Reelin in the Prefrontal Cortex

Animals that received stimulation exhibited a 43% increase in expression of reelin in the contralesional PFC compared to nonstimulated animals. Reelin is an extracellular glycoprotein that activates lipoprotein receptors, subsequently modulating synaptic function, learning, and memory. Experiments in heterozygous reelin knockout mice demonstrated reduced prefrontal dendritic spine density and associative learning deficits. Furthermore, reelin deficient mice have been shown to have reduced neurogenesis and reduced migration of SVZ-derived progenitors from the RIMS. Taken together, the increased reelin expression in the PFC of stimulated animals represents a molecular mechanism by which stimulation can augment plasticity and enhance the migration of neural progenitors as they travel along the RMS.

### Discussion

Based on the existence of projections to and from the NAcc, the brain region is believed to be a major node in the learning and memory system. The NAcc receives input from the hippocampal formation and the midbrain dopaminergic system, allowing memory and reinforcement information to converge. Furthermore, output projections from the NAcc allow direct and indirect influence on learning centers in PFC and motor execution centers in the brainstem, thereby facilitating the integration of learning with motor action. Consistent with these roles, phasic stimulation of the NAcc during the reinforcement period of a visuomotor spatial memory task was observed to enhance the behavioral performance of brain injured animals. Notably, the temporal specificity of the brain stimulation strategy was paramount, as continuous stimulation or stimulation at a different time point did not elicit the same effect. Under this framework, stimulating when an animal encounters a designated reward location could work to strengthen active synapses that lead to a correct response, altering the signal to noise ratio of the spatial learning and memory circuitry. Nevertheless, stimulated animals did not significantly improve in performance until day three and did not match uninjured animals until day seven. Therefore, stimulation may induce diverse effects on different time-scales that act in concert to promote cognitive recovery. In line with this, present histology and genomic data point to a multifaceted mechanism.

The immunohistochemistry analysis of stimulated animals revealed a striking increase in the presence neural progenitor cells in the PFC as well as an impressive increase in synaptic density in both the PFC and NAc. These observations can be explained by stimulation in the NAcc having not only a local effect that can modulate PFC through the dopaminergic system, but also the ability to modulate prefrontal activity patterns through antidromic activation of corticostriatal connections. This complements the influence of the NAcc as a central node in learning and memory, as stimulation of other sub-cortical structures, such as the anterior thalamic nucleus, has not been shown to have the same effect. Interestingly, a strong promotional effect in the contralesional cortex was also observed. This finding indicated that unilateral stimulation can affect interhemispheric interactions to modulate the balance of bilateral cortical recovery. A similar finding was reported in motor cortex of a stroke induced rodent model.

Molecular profiling revealed cellular alterations that provide another dimension for the observed behavioral enhancement and gross cellular reorganization. Enriched expression profiles of genes involved in neural structure, signaling, protection, migration, differentiation, and potentiation in all studied brain regions indicated that stimulation can substantially regulate widespread molecular changes to enhance signal processing efficiency and enable neural network recovery. These results strongly suggested that stimulation in the NAcc can augment mechanisms at both the molecular and systems level to facilitate an enhanced recovery from neurological deficits caused by TBI.

In summary, the above example provides evidence that temporally precise activation of the NAcc can augment intrinsic neuronal mechanisms to maximize behavioral outcomes and restore neurological deficits caused by TBI. Although the result was specific to a spatial memory task, the accumbens may be a promising candidate site for TBI intervention. The wide acceptance of brain stimulation for other indications makes this a particularly exciting and plausible mode of treatment. The rich connectivity of the NAcc has implicated this region in a number of learning, memory and motivational processes, all key characteristics needed for cognitive and motor recovery. Furthermore, the results from this study demonstrate that simulation in the accumbens can enhance healing and protective mechanisms, which may benefit the neural recovery process generally. It is also noteworthy that the above study was executed in a sub-acute phase of injury, not limiting the benefits of treatment to acute care. The findings of this investigation demonstrate that the post-injury period represents a major and underutilized opportunity to apply neuromodulatory intervention to optimize functional recovery.

While the invention is described through the above-described exemplary embodiments, it will be understood by those of ordinary skill in the art that modifications to, and variations of, the illustrated embodiments may be made without departing from the inventive concepts disclosed herein. Moreover, while the preferred embodiments are described in connection with various illustrative data structures, one skilled in the art will recognize that the system may be embodied using a variety of data structures. Furthermore, disclosed aspects, or portions of these aspects, may be combined in ways not specifically listed above. Accordingly, it is felt therefore that the scope of protection provided by this patent should not be viewed as limited by the above description, but rather should only be limited by the scope of the below claims.

1. A method for stimulating the brain of a patient to treat a medical condition, the method comprising:
positioning a stimulating device comprising electrical contacts configured to electrically stimulate locations associated with a patient's brain;
initiating a rehabilitation process to include the patient performing a task;
acquiring feedback from the patient at least while the patient is performing the task; and
generating, based on the acquired feedback, electrical stimulations to treat the medical condition of the patient.

2. The method of claim 1, wherein the locations are associated with a hippocampus, or a nucleus basalis, or a mammillary body, or a caudate, or a nucleus accumbens, or a combination thereof.

3. The method of claim 1, wherein the method further comprises transmitting, using the electrical contacts, the electrical stimulations in accordance with triggers provided by a capture system.

4. The method of claim 1, wherein the electrical stimulations include a plurality of electrical signal pulses.

5. The method of claim 4, wherein the electrical signal pulses include biphasic signal pulses, or monophasic signal pulses, or both.

6. The method of claim 4, wherein the plurality electrical signal pulses are defined by current amplitudes between 0 and 10 milli-Amperes, voltage amplitudes between 0 and 10 Volts, frequencies between 0 and 300 Hertz, pulse widths between 0 and 250 microseconds, durations between 0 and 10 seconds, and combinations thereof.

7. The method of claim 1, the method further comprising generating a report indicative of a patient performance.

8. A method for stimulating the brain of a patient to treat a medical condition, the method comprising:
positioning a stimulating device comprising electrical contacts configured to electrically stimulate a plurality of locations in a patient's brain;
initiating a rehabilitation process to include the patient performing a task;
providing, using the stimulating device, a first electrical stimulation to a first location in the patient's brain, the first electrical stimulation occurring at a first time point during the task;
acquiring, using a capture system, feedback from the patient while the patient is performing the task; and
providing, using the acquired feedback, a second electrical stimulation to a second location in the patient's brain, the second electrical stimulation occurring at a second time point relative to the first time point.

9. The method of claim 8, wherein the first electrical stimulation is provided at locations associated with a nucleus accumbens of the patient.

10. The method of claim 8, wherein the second electrical stimulation is provided at locations associated with a caudate of the patient.

11. The method of claim 8, the method further comprises triggering the stimulating device to transmit the first and second electrical stimulation using the electrical contacts.

12. The method of claim 8, wherein the first and second electrical stimulation includes a plurality of electrical signal pulses.

13. The method of claim 12, wherein the plurality electrical signal pulses are defined by current amplitudes between 0 and 10 milli-Amperes, voltage amplitudes between 0 and 10 Volts, frequencies between 0 and 300 Hertz, pulse widths between 0 and 250 microseconds, durations between 0 and 10 seconds, and combinations thereof.

14. The method of claim 8, wherein at least one of the first and second electrical stimulation includes biphasic signal pulses, or monophasic signal pulses, or both.

15. A system for stimulating the brain of a patient to treat a medical condition, the system comprising:
a stimulation system comprising electrical contacts configured to electrically stimulate locations associated with a patient's brain; and
a capture system, in communication with the stimulation system, comprising:
an input configured to receive feedback from the patient;
a processor at least configured to:
initiate a rehabilitation process to include the patient performing a task;
acquire, using the input, feedback from the patient;
generate an electrical stimulation based on the acquired feedback;
trigger the stimulation system to deliver the electrical stimulation to treat the medical condition of the patient.

16. The system of claim 15, wherein the locations are associated with a hippocampus, or a nucleus basalis, or a mammillary body, or a caudate, or a nucleus accumbens, or a combination thereof.

17. The system of claim 15, wherein the electrical stimulation includes a plurality of electrical signal pulses.

18. The system of claim 17, wherein the electrical signal pulses include biphasic signal pulses, or monophasic signal pulses, or both.

19. The system of claim 17, wherein the plurality electrical signal pulses are defined by current amplitudes between 0 and 10 milli-Amperes, voltage amplitudes between 0 and 10 Volts, frequencies between 0 and 300 Hertz, pulse widths between 0 and 250 microseconds, durations between 0 and 10 seconds, and combinations thereof.

20. The system of claim 15, wherein the processor is further configured to trigger the stimulation system to deliver stimulations to different brain regions at different points in time.

21. The system of claim 15, wherein the processor is further configured to adapt a second stimulation at a second time point using feedback acquired following a first stimulation delivered at a first time point.

22. The system of claim 15, wherein the stimulation system comprises an implantable device, or a wearable device, or both.

23. The system of claim 22, wherein the wearable device includes a far-field telemetry module for communicating with the capture system and a near-field telemetry module for communicating with the implantable device to deliver the electrical stimulation.

24. The system of claim 15, wherein the medical condition includes a brain injury (“TBI”) or a stroke.

25. The system of claim 15, wherein the processor is further configured to generate a report indicative of a patient performance.
